(19) United States
(12) Patent Application Publication
(10) Pub. No.: US 2008/0058309 A1
(43) Pub. Date: Mar. 6, 2008

(54) Novel Compounds 171

(75) Inventors: David Cheshire, Loughborough (GB); Simon Guile, Loughborough (GB); Toby Thompson, Loughborough (GB)

Correspondence Address: FISH & RICHARDSON P.C., P.O BOX 1022, MINNEAPOLIS, MN, 55440-1022, US

(73) Assignee: ASTRAZENECA AB

(21) Appl. No.: 11/828,577

(22) Filed: Jul. 26, 2007

Related U.S. Application Data
(60) Provisional application No. 60/833,675, filed on Jul. 27, 2006.

(51) Int. Cl. A61K 31/4709; A61K 31/55

(52) U.S. Cl. 514/210.21; 514/314; 546/167; 514/217.07

(57) ABSTRACT

The invention provides compounds of formula (I), processes for their preparation, pharmaceutical compositions containing them, a process for preparing the pharmaceutical compositions, and their use in therapy, wherein A, D, R¹, R², R³, R⁴, R⁵, R⁶, n, p and q are as defined in the specification.

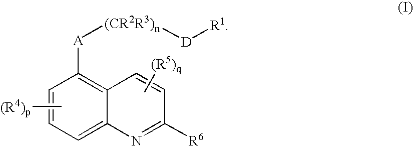

### CROSS REFERENCE TO RELATED APPLICATIONS

Pursuant to 35 USC §119(e), this application claims the benefit of prior U.S. Provisional Application 60/833,675, filed Jul. 27, 2006. The contents of this application is incorporated herein by reference in its entirety.

### TECHNICAL FIELD

The present invention relates to quinoline derivatives, processes for their preparation, pharmaceutical compositions containing them, a process for preparing pharmaceutical compositions, and their use in therapy.

### BACKGROUND

The P2X₇ receptor (previously known as P2Z receptor), which is a ligand-gated ion channel, is present on a variety of cell types, largely those known to be involved in the inflammatory/immune process, specifically, macrophages, mast cells and lymphocytes (T and B). Activation of the P2X₇ receptor by extracellular nucleotides, in particular adenosine triphosphate, leads to the release of interleukin-1β (IL-1β) and giant cell formation (macrophages/microglial cells), degranulation (mast cells) and proliferation (T cells) and apoptosis and L-selectin shedding (lymphocytes). P2X₇ receptors are also located on antigen-presenting cells (APC), keratinocytes, salivary acinar cells (parotid cells), hepatocytes and mesangial cells.

It would be desirable to make compounds effective as P2X₇ receptor antagonists for use in the treatment of inflammatory, immune or cardiovascular diseases, in the aetiologies of which the P2X₇ receptor may play a role.

P2X₇ antagonists comprising quinolinyl groups are known from WO2003/080579, WO 2004/106305 and WO2005/009968. In each of these disclosures it is necessary for the compounds to contain either a carbocyclyl-alkylamido or a heterocyclyl-alkylamido group. In contrast, the present invention provides compounds active as P2X₇ antagonists comprising an aryl- or heteroaryl-heteroalkylamido group.

### SUMMARY AND DETAILED DESCRIPTION

In accordance with the present invention, there is therefore provided a compound of formula (I), or a pharmaceutically acceptable salt thereof,

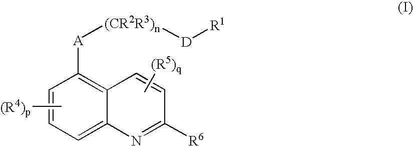

wherein

n is 1, 2 or 3;

when n is 1, A represents NHC(O) or NHC(S);

when n is 2 or 3, A represents NHC(O), C(O)NH, NHC(S) or C(S)NH;

D represents O, S or NR⁷, wherein R⁷ represents hydrogen, C₁₋₆ alkyl, C₁₋₆ hydroxyalkyl or C₁₋₆ haloalkyl;

R¹ represents a 6-10 membered aryl, or a 5-10 membered heteroaryl ring, which aryl or heteroaryl ring may be optionally substituted by one or more substituents independently selected from halogen, cyano, C₁₋₆ alkyl, C₁₋₆ hydroxyalkyl, C₁₋₆ haloalkyl, NR⁸R⁹, S(O)₀₋₂R¹⁰, S(O)₂NR¹¹R¹², C(O)NR¹³R¹⁴, CO₂R¹⁵, NR¹⁶S(O)₂R¹⁷, C(O)R¹⁸, NR¹⁹C(O)R²⁰, NR²¹C(O)NR²²R²³, NR²⁴S(O)₂NR²⁵R²⁶ and OR27;

within each grouping CR²R³, R² and R³ each independently represent hydrogen, halogen, C₁₋₃ alkyl, C₁₋₃ hydroxyalkyl or C₁₋₃ haloalkyl;

p represents 0, 1, 2 or 3; each R⁴ independently represents halogen, cyano, C₁₋₆ alkyl, C₁₋₆ hydroxyalkyl or C₁₋₆ haloalkyl;

q represents 0, 1 or 2; each R⁵ independently represents halogen, cyano, C₁₋₆ alkyl, C₁₋₆ hydroxyalkyl or C₁₋₆ haloalkyl;

R⁶ represents a group

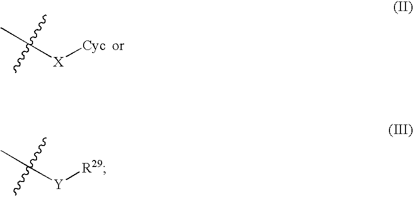

wherein X represents a bond, O, S, NR³⁰ or a C₁₋₄ alkylene which C₁₋₄ alkylene may be optionally substituted by one or more substituents independently selected from halogen, hydroxy and C₁₋₄ alkoxy;

Cyc represents a 3-8 membered carbocyclic or a 4-8 membered heterocyclic ring, which ring may be optionally substituted by one or more substituents R²⁸, wherein each substituent R²⁸ is independently selected from halogen, cyano, ═O, S(O)₂R³¹, C(O)R³², CO₂R³³, C(O)NR³⁴R³⁵, NR³⁶C(O)R³⁷, S(O)₂NR³⁸R³⁹, NR⁴⁰S(O)₂R⁴¹, OR⁴², SR⁴³, NR⁴⁴R⁴⁵, Z¹ or a C₁₋₆ alkyl group which C₁₋₆ alkyl group may be optionally substituted by one or more substituents independently selected from halogen, hydroxyl, C₁₋₆ alkoxy, NR⁴⁶R⁴⁷, S(O)₀₋₂, C(O)R⁴⁹, CO₂R⁵⁰, C(O)NR⁵¹R⁵², NR⁵³C(O)R⁵⁴ S(O)₂NR⁵⁵R⁵⁶, NR⁵⁷S(O)₂R⁵⁸ and Z²;

Y represents a bond, OC₁₋₆ alkylene, S(O)₀₋₂C₁₋₆alkylene, N(R⁵⁹)C₁₋₆alkylene or C₁₋₆ alkylene, wherein any alkylene group in Y may be optionally substituted by one or more substituents independently selected from halogen, hydroxy and C₁₋₄ alkoxy;

R²⁹ represents hydrogen, halogen, hydroxyl, C₁₋₆alkoxy, cyano, NR⁶⁰R⁶¹, S(O)₀₋₂R⁶², C(O)R⁶³, CO₂R⁶⁴, C(O)NR⁶⁵R⁶⁶, NR⁶⁷C(O)R⁶⁸, S(O)₂NR⁶⁹R⁷⁰, NR⁷¹S(O)₂R⁷² or Z³; with the proviso that when Y is a bond R²⁹ is not hydrogen;

R⁴², R⁴³, R⁴⁴ and R45 each independently represent hydrogen or a C₁₋₆ alkyl group which C₁₋₆ alkyl group may be optionally substituted by one or more substituents independently selected from halogen, hydroxyl, C₁₋₆ alkoxy, NR⁷³R⁷⁴, S(O)₀₋₂R⁷⁵, C(O)R⁷⁶, CO₂R⁷⁷, C(O)NR⁷⁸R⁷⁹, NR⁸⁰C(O)R⁸¹, S(O)₂NR⁸²R⁸³, NR⁸⁴S(O)₂R⁸⁵ or Z⁴, or R⁴⁴ and R⁴⁵, together with the nitrogen atom to which they are both attached, may form a 4-8 membered aliphatic heterocyclic ring, which heterocyclic ring may be optionally substituted by one or more substituents independently selected from halogen, hydroxy, C₁₋₆ alkyl, C₁₋₆ hydroxyalkyl and C₁₋₆ haloalkyl;

Z¹, Z², Z³ and Z⁴ each independently represent tetrazole, or a 5-6 membered heterocyclic ring, which heterocyclic ring is substituted by one or more substituents independently selected from hydroxy, amino, ═O, ═S, and which heterocyclic ring may further be optionally substituted by one or more substituents independently selected from halogen and C₁₋₆ alkyl;

R⁸, R⁹, R¹¹, R¹², R¹³, R¹⁴, R¹⁵, R¹⁶, R¹⁸, R¹⁹, R²⁰, R²¹, R²², R²³, R²⁴, R²⁵, R²⁶, R²⁷, R³⁰, R³², R³³, R³⁴, R³⁵, R³⁶, R³⁷, R³⁸, R³⁹, R⁴⁰ , R⁴⁶, R⁴⁷, R⁴⁹, R⁵⁰, R⁵¹, R⁵², R⁵³, R⁵⁴, R⁵⁵, R⁵⁶, R⁵⁷, R⁵⁹, R⁶⁰, R⁶¹, R⁶³, R⁶⁴, R⁶⁵, R⁶⁶, R⁶⁷, R⁶⁸, R⁶⁹, R⁷⁰, R⁷¹, R⁷³, R⁷⁴, R⁷⁶, R⁷⁷, R⁷⁸, R⁷⁹, R⁸⁰, R⁸¹, R⁸², R⁸³ and R⁸⁴ each independently represent hydrogen, C₁₋₆ alkyl, C₁₋₆ hydroxyalkyl or C₁₋₆haloalkyl; or any of R³ and R⁹, R¹¹ and R¹², R¹³ and R¹⁴, R²² and R²³, R²⁵ and R²⁶, R³⁴ and R³⁵, R³⁸ and R³⁹, R⁴⁶ and R⁴⁷, R⁵¹ and R⁵², R⁵⁵ and R⁵⁶, R⁶⁰ and R⁶¹, R⁶⁵ and R⁶⁶, R⁶⁹ and R⁷⁰, R⁷³ and R⁷⁴, R⁷⁸ and R⁷⁹, and R⁸² and R⁸³, together with the nitrogen atom to which they are both attached, may form a 4-8 membered aliphatic heterocyclic ring, which heterocyclic ring may be optionally substituted by one or more substituents independently selected from halogen, hydroxy, C₁₋₆ alkyl, C₁₋₆ hydroxyalkyl and C₁₋₆ haloalkyl; and

R¹⁰, R¹⁷, R³¹, R⁴¹, R⁴⁸, R⁵⁸, R⁶², R⁷², R⁷⁵ and R⁸⁵ each independently represent C₁₋₆ alkyl, C₁₋₆ hydroxyalkyl or C₁₋₆ haloalkyl; and

with the proviso that when R¹ is phenyl, D is O, n is 1, R² is hydrogen, R³ is hydrogen, A is NHC(O), p is 0, q is 0 and R⁶ is methyl, then the phenyl group R¹ must be substituted by at least one substituent other than C₁₋₄ alkyl, chlorine and methoxy.

It will be understood that certain compounds of the present invention may exist in solvated, for example hydrated, as well as unsolvated forms. It is to be understood that the present invention encompasses all such solvated forms. Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

In the context of the present specification the term ‘Carbocyclic’ denotes aliphatic or aromatic carbon rings; ‘Cycloalkyl’ denotes aliphatic carbon rings (i.e. saturated or partially saturated rings) for example cyclopropyl, cyclopentyl, cyclohexyl or cyclohexenyl; and ‘Aryl’ denotes aromatic carbon rings, for example phenyl or naphthyl. The term ‘Heterocyclic’ denotes aliphatic or aromatic rings comprising at least one heteroatom selected from the group comprising nitrogen, oxygen and sulfur; or an N-oxide thereof, or an S-oxide or S-dioxide thereof.

The term ‘Heteroaryl’ denotes aromatic rings comprising at least one heteroatom selected from the group comprising nitrogen, oxygen and sulfur; or an N-oxide thereof, or an S-oxide or S-dioxide thereof: examples of heteroaryl groups include furyl, thienyl, pyrrolyl, thiazolyl, pyrazolyl, oxazolyl, isoxazolyl, imidazolyl, pyridinyl, pyrimidinyl, pyrazinyl, pyridazinyl, indolyl, benzo[b]furyl (also known as benzfuryl), benzo[b]thienyl (also known as benz[b]thienyl, benzothienyl or benzo[b]thiophenyl), 2,3-dihydrobenz[b]thienyl (for example in a 1-dioxo-2,3-dihydrobenz[b]thienyl moiety), indazolyl, benzimidazolyl, benztriazolyl, benzoxazolyl, benzthiazolyl, 1,2,3-benzothiadiazolyl, an imidazopyridinyl (such as imidazo[1,2-a]pyridinyl), a thienopyridinyl (for example thieno[3,2-b]pyridin-6-yl, thieno[2,3-c]pyridin-2-yl or thieno[3,2-c]pyridin-2-yl), thieno[3,2-d]pyrimidin-6-yl, 1,2,3-benzoxadiazolyl, benzo[1,2,3]thiadiazolyl, 2,1,3-benzothiadiazolyl, benzofurazan (also known as 2,1,3-benzoxadiazolyl), quinoxalinyl, a pyrazolopyridine (for example 1H-pyrazolo[3,4-b]pyridinyl), quinolinyl, isoquinolinyl, a naphthyridinyl (for example [1,6]naphthyridinyl or [1,8]naphthyridinyl) or a benzothiazinyl; or an N-oxide thereof, or an S-oxide or S-dioxide thereof. The term ‘Aliphatic heterocyclic ring’ denotes a saturated or partially saturated monocyclic ring comprising at least one heteroatom selected from the group comprising nitrogen, oxygen and sulphur: or an N-oxide thereof, or an S-oxide or S-dioxide thereof: for example pyrrolidinyl, piperidinyl, piperazinyl, morpholinyl, homopiperazinyl, homopiperidinyl and azetidinyl.

In the context of the present specification alkyl groups and moieties may be straight or branched chain and include, for example, methyl, ethyl, n-propyl, iso-propyl, n-butyl, iso-butyl or tert-butyl. Haloalkyl moieties may contain one or more (e.g. one, two, three, four, five or six) halogen atoms. In the present specification halogen is for example, fluorine, chlorine or bromine. Hydroxyalkyl moieties may contain one or more (e.g. one, two or three) hydroxyl groups. In general, a hydroxyl moiety will not be attached to a carbon atom which is adjacent to a nitrogen atom. In the context of the present specification, where it is stated that a group may be optionally substitued by one or more substituents the group may be unsubstituted or substituted; when substituted the group will generally be substitued with one, two or three substituents.

In the present invention n represents 1, 2 or 3. In an embodiment of the invention, n represents 1 or 2. In another embodiment, n represents 1.

When n is 1, A represents NHC(O) or NHC(S); and when n is 2 or 3, A represents NHC(O), C(O)NH, NHC(S) or C(S)NH.

In an embodiment of the invention, when n is 1, A represents NHC(O); and when n is 2 or 3, A represents NHC(O) or C(O)NH.

In an embodiment of the invention, A represents NHC(O).

In the present invention D represents O, S or NR⁷, wherein R⁷ represents hydrogen, C₁₋₆ alkyl, C₁₋₆ hydroxyalkyl or C₁₋₆ haloalkyl.

In an embodiment of the invention, D represents NR⁷ and R⁷ represents hydrogen, C₁₋₄ alkyl, C₁₋₄ hydroxyalkyl or C₁₋₄ haloalkyl. In a further aspect of this embodiment, D represents NR⁷, and R⁷ represents hydrogen or C₁₋₄ alkyl.

R¹ represents a 6-10 membered aryl, or a 5-10 membered heteroaryl ring, which aryl or heteroaryl ring may be optionally substituted by one or more substituents independently selected from halogen, cyano, C₁₋₆ alkyl, C₁₋₆ hydroxyalkyl, C₁₋₆ haloalkyl, NR⁸R⁹, S(O)₀₋₂R¹⁰, S(O)₂NR¹¹R¹², C(O)NR¹³R¹⁴, CO₂R¹⁵, NR¹⁶S(O)₂R¹⁷, C(O)R¹⁸, NR¹⁹C(O)R²⁰, NR²¹C(O)NR²²R²³, NR²⁴S(O)₂NR²⁵R²⁶ and OR²⁷.

In an embodiment of the invention, R¹ represents phenyl, which phenyl may be optionally substituted by one or more substituents independently selected from halogen, cyano, C₁₋₄ alkyl, C₁₋₄ hydroxyalkyl, C₁₋₄ haloalkyl, NR⁸R⁹, S(O)₀₋₂R¹⁰, S(O)₂NR¹¹R¹², C(O)NR¹³R¹⁴, CO₂R¹⁵, NR¹⁶S(O)₂R¹⁷, C(O)R¹⁸, NR¹⁹C(O)R²⁰, NR²¹C(O)NR²²R²³, NR²⁴S(O)₂NR²⁵R²⁶ and OR²⁷.

In an embodiment of the invention, R¹ represents phenyl, which phenyl may be optionally substituted by one or more substituents independently selected from halogen, cyano, C₁₋₄ alkyl, C₁₋₄ hydroxyalkyl, C₁₋₄ haloalkyl, hydroxyl, C₁₋₄ alkoxy, amino, C₁₋₄alkylamino and di-(C₁₋₄alkyl)amino.

In an embodiment of the invention R¹ represents an optionally substituted 5-10 membered heteroaryl ring. When R¹ represents a 5-10 membered heteroaryl ring, examples of heteroaryl rings include pyridinyl, pyrimidinyl, pyrazinyl and pyridazinyl.

Within each grouping CR²R³, R² and R³ each independently represent hydrogen, C₁₋₃ alkyl, C₁₋₃ hydroxyalkyl or C₁₋₃ haloalkyl. In an embodiment of the invention, each R² and R³ independently represent hydrogen or C₁₋₃ alkyl. In an embodiment of the invention, each R² and R³ independently represent hydrogen.

Each R⁴ independently represents halogen, cyano, C₁₋₆ alkyl, C₁₋₆ hydroxyalkyl or C₁₋₆ haloalkyl. In an embodiment of the invention, each R⁴ independently represents halogen or C₁₋₄ alkyl. In an embodiment of the invention, each R⁴ independently represents halogen (e.g. fluorine, chlorine or bromine).

In the present invention p is 0, 1, 2 or 3. In an embodiment of the invention p is 0 or 1. In an embodiment of the invention p is 1.

Each R⁵ independently represents halogen, cyano, C₁₋₆ alkyl, C₁₋₆ hydroxyalkyl or C₁₋₆ haloalkyl. In an embodiment of the invention, each R⁵ independently represents halogen or C₁₋₄ alkyl. In an embodiment of the invention, each R⁵ independently represents halogen (e.g. fluorine, chlorine or bromine).

In the present invention q is 0, 1 or 2. In an embodiment of the invention q is 0 or 1. In an embodiment of the invention q is 0.

In an embodiment of the invention, R⁶ represents a group

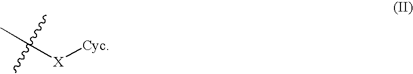

In an embodiment of the invention, X represents a bond.

In an embodiment of the invention, X represents NR³⁰. In a further aspect of this embodiment, R³⁰ represents hydrogen or C₁₋₄ alkyl.

In an embodiment of the invention, Cyc represents a 4-8 membered heterocyclic ring, which heterocyclic ring may be optionally substituted by one or more substituents R²⁸.

Examples of heterocyclic rings that Cyc may represent include, pyrrolidinyl, piperidinyl, piperazinyl, morpholinyl, homopiperazinyl, homopiperidinyl, azetidinyl, triazinyl, pyrazolyl and triazolyl. In an embodiment of the invention Cyc represents an optionally substituted pyrrolidinyl or morpholinyl ring.

In the present invention each substituent R²⁸ independently represents halogen, cyano, ═O, S(O)₂R³¹, C(O)R³², CO₂R³³, C(O)NR³⁴R³⁵, NR³⁶C(O)R³⁷, S(O)₂NR³⁸R³⁹, NR⁴⁰S(O)₂R⁴¹, OR⁴², SR⁴³, NR⁴⁴R⁴⁵, Z¹ or a C₁₋₆ alkyl group which C₁₋₆ alkyl group may be optionally be substituted by one or more substituents independently selected from halogen, hydroxyl, C₁₋₆ alkoxy, NR⁴⁶R⁴⁷, S(O)₀₋₂R⁴⁸, C(O)R⁴⁹, CO₂R⁵⁰, C(O)NR⁵¹R⁵², NR⁵³C(O)R⁵⁴, S(O)₂NR⁵⁵R⁵⁶, NR⁵⁷S(O)₂R⁵⁸ and Z².

In an embodiment of the invention, each substituent R²⁸ is independently selected from halogen, OR⁴², NR⁴⁴R⁴⁵ or a C₁₋₄ alkyl group, which C₁₋₄ alkyl group may be optionally substituted by one or more substituents independently selected from halogen, hydroxyl, C₁₋₄ alkoxy, NR⁴⁶R⁴⁷ and C(O)NR⁵¹R⁵². In a further aspect of this embodiment, R⁴⁶, R⁴⁷, R⁵¹ and R⁵² each independently represent hydrogen or a C₁₋₄ alkyl group optionally substituted by one or more substituents independently selected from halogen and hydroxyl.

In the present invention R⁴², R⁴³, R⁴⁴ and R⁴⁵ each independently represent hydrogen or a C₁₋₆ alkyl group which C₁₋₆ alkyl group may be optionally substituted by one or more substituents independently selected from halogen, hydroxyl, C₁₋₆ alkoxy, NR⁷³R⁷⁴, S(O)₀₋₂R⁷⁵, C(O)R⁷⁶, CO₂R⁷⁷, C(O)NR⁷⁸R⁷⁹, NR⁸⁰C(O)R⁸¹, S(O)₂NR⁸²R⁸³, NR⁸⁴S(O)₂R⁸⁵ or Z⁴, or R⁴⁴ and R⁴⁵, together with the nitrogen atom to which they are both attached, may form a 4-8 membered aliphatic heterocyclic ring, which heterocyclic ring may be optionally substituted by one or more substituents independently selected from halogen, hydroxy, C₁₋₆ alkyl, C₁₋₆ hydroxyalkyl and C₁₋₆ haloalkyl.

In an embodiment of the invention, R⁴², R⁴³, R⁴⁴ and R⁴⁵ each independently represent hydrogen or a C₁₋₄ alkyl group which C₁₋₄ alkyl group may be optionally substituted by one or more substituents independently selected from halogen, hydroxyl, C₁₋₄ alkoxy, NR⁷³R⁷⁴ and C(O)NR⁷⁸R⁷⁹. In a further aspect of this embodiment, R⁷³, R⁷⁴, R⁷³ and R⁷⁹ each independently represent hydrogen or a C₁₋₄ alkyl group, which C₁₋₄ alkyl group may be optionally substituted by one or more substituents independently selected from halogen and hydroxyl.

In an embodiment of the invention, each substituent R²⁸ is independently selected from halogen, OR⁴², NR⁴⁴R⁴⁵ or a C₁₋₄ alkyl group, which C₁₋₄ alkyl group may be optionally substituted by one or more substituents independently selected from halogen, hydroxyl, C₁₋₄ alkoxy, NR⁴⁶R⁴⁷ and C(O)NR⁵¹R⁵²;

R⁴², R⁴⁴ and R⁴⁵ each independently represent hydrogen or a C₁₋₄ alkyl group which C₁₋₄ alkyl group may be optionally substituted by one or more substituents independently selected from halogen, hydroxyl, C₁₋₄ alkoxy, NR⁷³R⁷⁴ and C(O)NR⁷⁸R⁷⁹; and R⁴⁶, R⁴⁷, R⁵¹, R⁵², R⁷³, R⁷⁴, R⁷⁸ and R⁷⁹ each independently represent hydrogen or a C₁₋₄ alkyl group which C₁₋₄ alkyl group may be optionally substituted by one or more substituents independently selected from halogen and hydroxyl.

In an embodiment of the invention, each substituent R²⁸ is independently selected from hydroxy, methyl, ethyl, —NH₂, —NHCH₃, —NHCH₂CH₂OH, —CH₂C(O)OH, —NHCH₂C(O)OH, —NHCH₂CH₂C(O)OH, —CH₂NHCH₃, —CH₂NHCH₂CH₂OH, —NHCH₂C(O)NHCH₃, —NHCH(CH₃)CH₂OH, —N(CH₃)CH₂CH(OH)CH₂OH, —CH₂CH(OH)CH₂OH, —CH(CH₃)CH(OH)CH₂OH, —CH₂C(O)NHCH₃, —CH₂CH(OH)CH₂OCH₃ and —CH₂C(O)N(CH₃)CH₂CH₂OH. In a further aspect of this embodiment, Cyc represents a pyrrolidinyl or morpholinyl ring.

In an embodiment of the invention, R⁶ represents a group selected from

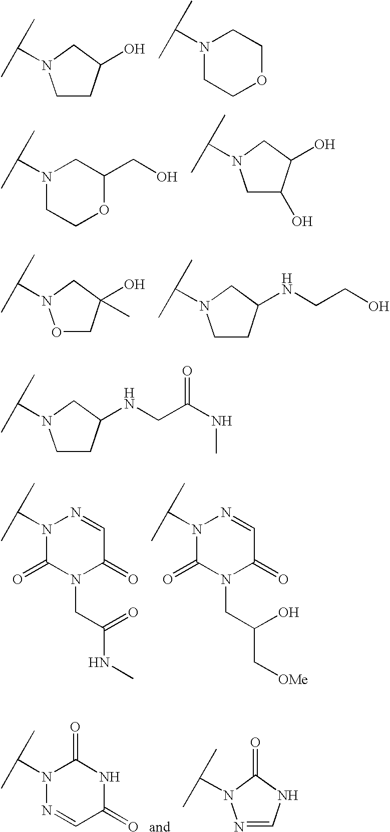

In an embodiment of the invention, R⁶ represents a group

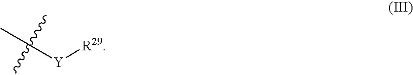

In an embodiment of the invention, Y represents a bond, OC₁₋₄ alkylene, N(R⁵⁹)C₁₋₄alkylene or C₁₋₄ alkylene, wherein any alkylene group in Y may be optionally substituted by one or more substituents independently selected from halogen, hydroxy and C₁₋₄ alkoxy. In a further aspect of this embodiment R⁵⁹ represents hydrogen or C₁₋₄ alkyl.

In the present invention, R²⁹ represents hydrogen, halogen, hydroxyl, C₁₋₆alkoxy, cyano, NR⁶⁰R⁶¹, S(O)₀₋₂R⁶², C(O)R⁶³, CO₂R⁶⁴, C(O)NR⁶⁵R⁶⁶, NR⁶⁷C(O)R⁶⁸, S(O)₂NR⁶⁹R⁷⁰, NR⁷¹S(O)₂R⁷² or Z³; with the proviso that when Y is a bond R²⁹ is not hydrogen.

In an embodiment of the invention R²⁹ represents hydrogen, halogen, hydroxyl, C₁₋₄alkoxy, NR⁶⁰R⁶¹ and C(O)NR⁶⁵R⁶⁶. In a further aspect of this embodiment R⁶⁰, R⁶¹, R⁶⁵ is and R⁶⁶ each independently represent hydrogen or a C₁₋₄ alkyl group optionally substituted by one or more substituents independently selected from halogen and hydroxyl.

Z¹, Z², Z³ and Z⁴ each independently represent tetrazole, or a 5-6 membered heterocyclic ring, which heterocyclic ring is substituted by one or more substituents independently selected from hydroxy, amino, ═O, ═S, and which heterocyclic ring may further be optionally substituted by one or more substituents independently selected from halogen and C₁₋₆ alkyl. Examples of groups that Z¹, Z², Z³ and Z⁴ may represent include:

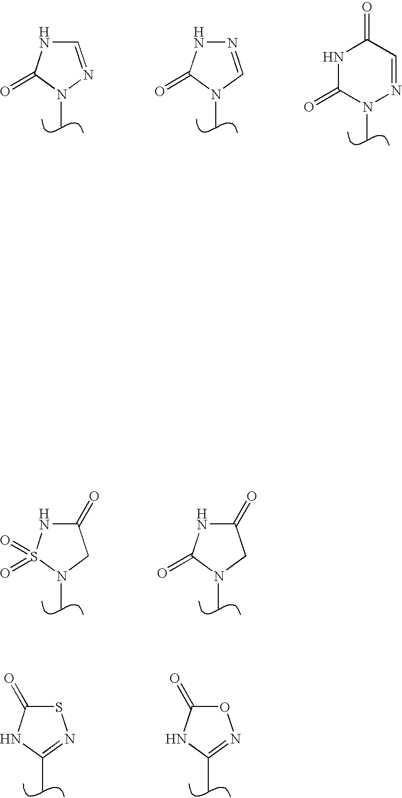

In an embodiment of the invention, R⁸, R⁹, R¹¹, R¹², R¹³, R¹⁴, R¹⁵, R¹⁶, R¹⁸, R¹⁹, R²⁰, R²¹, R²², R²³, R²⁴, R²⁵, R²⁶ and R²⁷ each independently represent hydrogen or C₁₋₄ alkyl. In an embodiment of the invention, R¹⁰ and R¹⁷ each independently represent C₁₋₄ alkyl.

In an embodiment of the invention, R³⁰ represents hydrogen or C₁₋₄ alkyl.

In an embodiment of the invention, R³², R³³, R³⁴, R³⁵, R³⁶, R³⁷, R³⁸, R³⁹, R⁴⁰, R⁴⁶, R⁴⁷, R⁴⁹, R⁵⁰, R⁵¹, R⁵², R⁵³, R⁵⁴, R⁵⁵, R⁵⁶ and R⁵⁷ each independently represent hydrogen or C₁₋₄ alkyl which C₁₋₄ alkyl group may be optionally substituted by one or more substituents independently selected from halogen and hydroxyl. In an embodiment of the invention, R³¹, R⁴¹, R⁴⁸ and R⁵⁸ each independently represent C₁₋₄ alkyl which C₁₋₄ alkyl group may be optionally substituted by one or more substituents independently selected from halogen and hydroxyl.

In an embodiment of the invention, R⁵⁹ represents hydrogen or C₁₋₄ alkyl.

In an embodiment of the invention, R⁶⁰, R⁶¹, R⁶³, R⁶⁴, R⁶⁵, R⁶⁶, R⁶⁷, R⁶⁸, R⁶⁹, R⁷⁰ and R⁷¹ each independently represent hydrogen or C₁₋₄ alkyl which C₁₋₄ alkyl group may be optionally substituted by one or more substituents independently selected from halogen and hydroxyl. In an embodiment of the invention, R⁶² and R⁷² each independently represent C₁₋₄ alkyl which C₁₋₄ alkyl group may be optionally substituted by one or more substituents independently selected from halogen and hydroxyl.

In an embodiment of the invention, R⁷³, R⁷⁴, R⁷⁶, R⁷⁷, R⁷⁸, R⁷⁹, R⁸⁰, R⁸¹, R⁸², R⁸³ and R⁸⁴ each independently represent hydrogen or C₁₋₄ alkyl which C₁₋₄ alkyl group may be optionally substituted by one or more substituents independently selected from halogen and hydroxyl. In an embodiment of the invention, R⁷⁵ and R⁸⁵ each independently represent C₁₋₄ alkyl which C₁₋₄ alkyl group may be optionally substituted by one or more substituents independently selected from halogen and hydroxyl.

In a further aspect, the present invention provides a compound of formula (IA), or a pharmaceutically acceptable salt thereof,

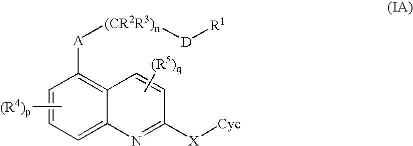

wherein

n is 1;

A represents NHC(O);

D represents O, S or NR⁷, wherein R⁷ represents hydrogen or C₁₋₄ alkyl;

R¹ represents phenyl, which phenyl may be optionally substituted by one or more substituents independently selected from halogen, cyano, nitro, C₁₋₄ alkyl, C₁₋₄ hydroxyalkyl, C₁₋₄ haloalkyl, NR⁸R⁹, S(O)₀₋₂R¹⁰, S(O)₂NR¹¹R¹², C(O)NR¹³R¹⁴, CO₂R¹⁵, NR¹⁶S(O)₂R¹⁷, C(O)R¹⁸, NR¹⁹C(O)R²⁰, NR²¹C(O)NR²²R²³, NR²⁴S(O)₂NR²⁵R²⁶ and OR²⁷;

R² and R³ each independently represent hydrogen or C₁₋₃ alkyl;

p represents 0 or 1; R⁴ represents halogen or C₁₋₄ alkyl;

q represents 0 or 1; R⁵ represents halogen or C₁₋₄ alkyl;

X represents a bond,

Cyc represents a 4-8 membered heterocyclic ring, which heterocyclic ring may be optionally substituted by one or more substituents R²⁸;

each substituent R²⁸ is independently selected from halogen, OR⁴², NR⁴⁴R⁴⁵ or a C₁₋₄ alkyl group, which C₁₋₄ alkyl group may be optionally substituted by one or more substituents independently selected from halogen, hydroxyl, C₁₋₄ alkoxy, NR⁴⁶R⁴⁷ and C(O)NR⁵¹R⁵²;

R⁴², R⁴⁴ and R⁴⁵ each independently represent hydrogen or a C₁₋₄ alkyl group which C₁₋₄ alkyl group may be optionally substituted by one or more substituents independently selected from halogen, hydroxyl, C₁₋₄ alkoxy, NR⁷³R⁷⁴ and C(O)NR⁷⁸R⁷⁹;

R⁴⁶, R⁴⁷, R⁵¹, R⁵², R⁷³, R⁷⁴, R⁷⁸ and R⁷⁹ each independently represent hydrogen or a C₁₋₄ alkyl group which C₁₋₄ alkyl group may be optionally substituted by one or more substituents independently selected from halogen and hydroxyl;

R⁸, R⁹, R¹¹, R¹², R¹³, R¹⁴, R¹⁵, R¹⁶, R¹⁸, R¹⁹, R²⁰, R²¹, R²², R²³, R²⁴, R²⁵, R²⁶ and R²⁷ each independently represent hydrogen or C₁₋₄ alkyl; and

R¹⁰ and R¹⁷ each independently represent C₁₋₄ alkyl.

In a further aspect, the present invention provides a compound of formula (IB), or a pharmaceutically acceptable salt thereof,

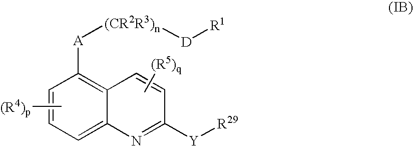

wherein

n is 1;

A represents NHC(O);

D represents O, S or NR⁷, wherein R⁷ represents hydrogen or C₁₋₄ alkyl;

R¹ represents phenyl, which phenyl may be optionally substituted by one or more substituents independently selected from halogen, cyano, nitro, C₁₋₄ alkyl, C₁₋₄ hydroxyalkyl, C₁₋₄ haloalkyl, NR⁸R⁹, S(O)₀₋₂R¹⁰, S(O)₂NR¹¹R¹², C(O)NR¹³R¹⁴, CO₂R¹⁵, NR¹⁶S(O)₂R¹⁷, C(O)R¹⁸, NR¹⁹C(O)R²⁰, NR²¹C(O)NR²²R²³, NR²⁴S(O)₂NR²⁵R²⁶ and OR²⁷;

R² and R³ each independently represent hydrogen or C₁₋₃ alkyl;

p represents 0 or 1; R⁴ represents halogen or C₁₋₄ alkyl;

q represents 0 or 1; R⁵ represents halogen or C₁₋₄ alkyl;

Y represents a bond, OC₁₋₄alkylene, N(R⁵⁹)C₁₋₄alkylene or C₁₋₄ alkylene, wherein any alkylene group in Y may be optionally substituted by one or more substituents independently selected from halogen, hydroxy and C₁₋₄ alkoxy;

R⁵⁹ represents hydrogen or C₁₋₄ alkyl;

R²⁹ represents hydrogen, halogen, hydroxyl, C₁₋₄alkoxy, NR⁶⁰R⁶¹ and C(O)NR⁶⁵R⁶⁶;

with the proviso that when Y is a bond R²⁹ is not hydrogen; and

R⁶⁰, R⁶¹, R⁶⁵ and R⁶⁶ each independently represent hydrogen or a C₁₋₄ alkyl group which C₁₋₄ alkyl group may be optionally substituted by one or more substituents independently selected from halogen and hydroxyl;

R⁸, R⁹, R¹¹, R¹², R¹³, R¹⁴, R¹⁵, R¹⁶, R¹⁸, R¹⁹, R²⁰, R²¹, R²², R²³, R²⁴, R²⁵, R²⁶ and R²⁷ each independently represent hydrogen or C₁₋₄ alkyl;

R¹⁰ and R¹⁷ each independently represent C₁₋₄ alkyl; and

with the proviso that when R¹ is phenyl, D is 0, n is 1, R² is hydrogen, R³ is hydrogen, A is NHC(O), p is 0, q is 0 and YR²⁹ represents methyl, then the phenyl group R¹ must be substituted by at least one substituent other than C₁₋₄ alkyl, chlorine and methoxy.

Pharmaceutically acceptable salts of a compound of formula (I) include, but are not limited to base salts such as an alkali metal salt for example sodium or potassium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. Where the compound is sufficiently basic, suitable salts include acid addition salts such as a hydrochloride, hydrobromide, phosphate, acetate, fumarate, maleate, tartrate, citrate, oxalate, methanesulphonate or p-toluenesulphonate salt. There may be more than one cation or anion depending on the number of charged functions and the valency of the cations or anions.

In an embodiment of the invention, the compound of formula (I) is selected from:

N-[6-Chloro-2-[(3R)-3-hydroxy-1-pyrrolidinyl]-5-quinolinyl]-2-(phenylamino)-acetamide,

N-[6-Chloro-2-[(3R)-3-hydroxy-1-pyrrolidinyl]-5-quinolinyl]-2-[(3-chlorophenyl)-amino]-acetamide,

N-[6-Chloro-2-[(3R)-3-hydroxy-1-pyrrolidinyl]-5-quinolinyl]-2-[(4-chlorophenyl)amino]-acetamide,

N-[6-Chloro-2-[(3R)-3-hydroxy-1-pyrrolidinyl]-5-quinolinyl]-2-[(2-chlorophenyl)amino]-acetamide,

N-[6-Chloro-2-[(3R)-3-hydroxy-1-pyrrolidinyl]-5-quinolinyl]-2-phenoxy-acetamide,

N-[6-Chloro-2-[(3R)-3-hydroxy-1-pyrrolidinyl]-5-quinolinyl]-2-(phenylthio)-acetamide,

N-[6-Chloro-2-[(3R)-3-hydroxy-1-pyrrolidinyl]-5-quinolinyl]-2-[(3-cyanophenyl)-amino]-acetamide,

N-[6-Chloro-2-[(3R)-3-hydroxy-1-pyrrolidinyl]-5-quinolinyl]-2-[(4-cyanophenyl)amino]-acetamide,

N-[6-Chloro-2-[(3R)-3-hydroxy-1-pyrrolidinyl]-5-quinolinyl]-2-[(3-fluorophenyl)amino]-acetamide,

N-[6-Chloro-2-[(3R)-3-hydroxy-1-pyrrolidinyl]-5-quinolinyl]-2-[(4-fluorophenyl)amino]-acetamide,

N-[6-Chloro-2-[(3R)-3-hydroxy-1-pyrrolidinyl]-5-quinolinyl]-2-[(3,4-difluorophenyl)amino]-acetamide,

N-[6-Chloro-2-[(3R)-3-hydroxy-1-pyrrolidinyl]-5-quinolinyl]-2-[(3,4-difluorophenyl)amino]-propanamide,

N-[6-Chloro-2-(1,2-dihydroxypropyl)-5-quinolinyl]-2-[(4-fluorophenyl)amino]-acetamide,

N-(6-Chloro-2-morpholin-4-yl-quinolin-5-yl)-2-(4-fluoro-phenylamino)-acetamide,

N-[6-Chloro-2-(1H-pyrazol-3-yl)-quinolin-5-yl]-2-(4-fluoro-phenylamino)-acetamide or a pharmaceutically acceptable salt thereof.

The present invention further provides a process for the preparation of a compound of formula (J) as defined above, or a pharmaceutically acceptable salt thereof, which comprises either

(a) reacting a compound of formula

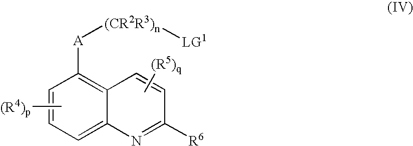

with a compound of formula
HD-R¹ (V)

wherein LG¹ represents a leaving group such as a halogeno or sulphonyloxy group (e.g. a chloro, bromo, iodo, trifluoromethanesulphonyloxy, methanesulphonyloxy or paratoluenesulphonyloxy group) and A, D, R¹, R², R³, R⁴, R⁵, R⁶, n, p and q are as defined in formula (I); or

(b) reacting a compound of formula

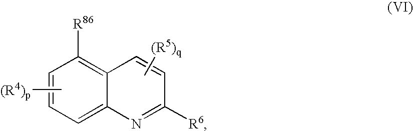

with a compound of formula

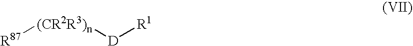

wherein one of R⁸⁶ and R⁸⁷ represents NH₂ and the other of R⁸⁶ and R⁸⁷ represents CO₂H, COF, COBr or COCl, and D, R¹, R², R³, R⁴, R⁵, R⁶, n, p and q are as defined in formula (I); or

(c) reacting a compound of formula

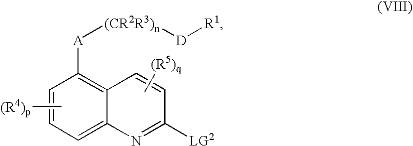

with a compound of formula

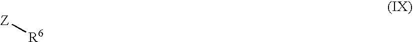

wherein LG² represents a leaving group such as a halogeno or sulphonyloxy group (e.g. a chloro, bromo, iodo, trifluoromethanesulphonyloxy, methanesulphonyloxy or paratoluenesulphonyloxy group), A, D, R¹, R², R³, R⁴, R⁵, R⁶, n, p and q are as defined in formula (I), and z either represents hydrogen when R⁶ is attached to z at a heteroatom, otherwise when R⁶ is attached to z at a carbon atom, z represents a metallic, organometallic or organosilicon group (e.g. copper, lithium, an organoboron group such as B(OH)₂, B(OⁱPr)₂, BEt₂ or a boronic acid pinacol cyclic ester, or an organotin group such as SnMe₃ or SnBu₃, or an organosilicon group such as Si(Me)F₂);

and optionally after (a), (b) or (c), carrying out one or more of the following:
converting the compound to a further compound of the invention
forming a pharmaceutically acceptable salt of the compound.

In process (a), the reaction is conveniently carried out in an organic solvent such as N-methylpyrrolidinone, acetonitrile or N,N-dimethylformamide, optionally in the presence of a base such as potassium carbonate, triethylamine or diisopropylethylamine, and at a temperature in the range from 25° C. to 180° C., in particular 80° C. to 150° C., either in a microwave or under conventional thermal conditions.

In process (b), the reaction may conveniently be carried out in the presence of a suitable coupling reagent, such as bromo-tris-pyrrolidino-phosphonium hexafluorophosphate (PyBroP) or dicyclohexylcarbodiimide and 1-hydroxybenzotriazole, in the presence of a base such as triethylamine, N-methylmorpholine, diisopropylethylamine or potassium carbonate, in a solvent such as dichloromethane, N-methylpyrrolidinone, N,N-dimethylformamide or tetrahydrofuran, and at a temperature in the range from 0° C. to 150° C., in particular 25° C. to 100° C.

In process (c), when z represents hydrogen, the reaction may conveniently be carried out in an organic solvent such as N-methylpyrrolidinone, acetonitrile or N,N-dimethylformamide, optionally in the presence of a base such as potassium carbonate or triethylamine, and at a temperature in the range from 25° C. to 180° C., in particular 50° C. to 120° C., either in a microwave or under conventional thermal conditions.

In process (c), when z represents a metallic or organometallic group, the reaction may conveniently be carried out in the presence of a catalyst such as tetrakis(triphenylphosphine)palladium(0), palladium(II) chloride or dichlorobis(triphenylphosphine)palladium(II), in the presence of a solvent such as tetrahydrofuran, 1,4-dioxane, 1,2-dimethoxyethane, toluene, methanol, or water, in the presence of a base such as sodium carbonate or potassium carbonate, and at a temperature in the range 10 to 250° C., preferably in the range 60 to 120° C.

Compounds of formula (IV) may be prepared by reacting a compound of formula (VI) as described in (b) above with a compound of formula

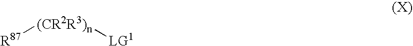

wherein LG¹ is as defined in formula (IV), R⁸⁷ is as defined in formula (VII), and R², R³ and n are as defined in formula (I), optionally in the presence of a suitable coupling reagent, such as bromo-tris-pyrrolidino-phosphonium hexafluorophosphate (PyBroP) or dicyclohexylcarbodiimide and 1-hydroxybenzotriazole, in the presence of a base such as triethylamine, N-methylmorpholine, diisopropylethylamine or potassium carbonate, in a solvent such as dichloromethane, N-methylpyrrolidinone, N,N-dimethylformamide, acetone or tetrahydrofuran, and at a temperature in the range from 0° C. to 150° C., in particular 25° C. to 100° C.

Alternatively, compounds of formula (IV) may be prepared by reacting a compound of formula

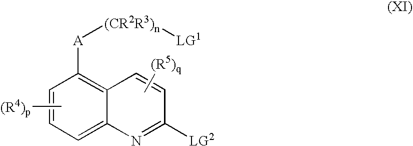

with a compound of formula

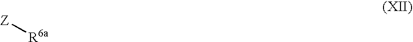

wherein R^{6a} either represents a group of formula R⁶ as defined in formula (I) or a precursor group that may be converted to R⁶ by standard chemical transformation, z is as defined in formula (IX), LG² is as defined in formula (VIII), LG¹ is as defined in formula (IV), and A, R², R³, R⁴, R⁵, n, p and q are as defined in formula (I). When R^{6a} is a precursor group to R⁶, the reaction of (XI) and (XII) is followed by a standard chemical transformation to convert the precursor group to R⁶ (e.g. dihydroxylation of an alkene). When z represents hydrogen and R^{6a} is attached to z at a heteroatom, the reaction between a compound of formula (XI) and a compound of formula (XII) may conveniently be carried out in an organic solvent such as N-methylpyrrolidinone, acetonitrile or N,N-dimethylformamide, optionally in the presence of a base such as potassium carbonate or triethylamine, and at a temperature in the range from 25° C. to 180° C., in particular 50° C. to 120° C., either in a microwave or under conventional thermal conditions. When z represents a metallic or organometallic group such as an organoboron group (e.g. B(OH)₂ or a boronic acid pinacol cyclic ester) or an organotin group (e.g. SnBu₃), and R^{6a} is attached to z at a carbon atom, the reaction between a compound of formula (XI) and a compound of formula (XII) may conveniently be carried out in the presence of a catalyst such as tetrakis(triphenylphosphine)palladium(0), palladium(II) chloride or dichlorobis(triphenylphosphine)palladium(II), in the presence of a solvent such as tetrahydrofuran, 1,4-dioxane, 1,2-dimethoxyethane, toluene, methanol, or water, in the presence of a base such as sodium carbonate or potassium carbonate, and at a temperature in the range 10 to 250° C., preferably in the range 60 to 120° C.

Compounds of formula (VI) may be prepared by reacting a compound of formula

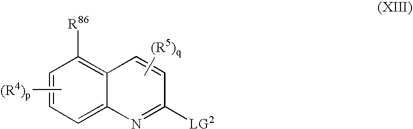

with a compound of formula (XII), wherein LG² is as defined in formula (VIII), R⁸⁶ is as defined in formula (VI), and R⁴, R⁵, p and q are as defined in formula (I). When R^{6a} is a precursor group to R⁶, the reaction of (XIII) and (XII) is followed by a standard chemical transformation to convert the precursor group to R⁶ (e.g. dihydroxylation of an alkene). When z represents hydrogen and R^{6a} is attached to z at a heteroatom, the reaction between a compound of formula (XIII) and a compound of formula (XII) may conveniently be carried out in an organic solvent such as N-methylpyrrolidinone, acetonitrile or N,N-dimethylformamide, optionally in the presence of a base such as potassium carbonate or triethylamine, and at a temperature in the range from 25° C. to 180° C., in particular 50° C. to 120° C., either in a microwave or under conventional thermal conditions. When z represents a metallic or organometallic group such as an organoboron group (e.g. B(OH)₂ or a boronic acid pinacol cyclic ester) or an organotin group (e.g. SnBu₃), and R^{6a} is attached to z at a carbon atom, the reaction between a compound of formula (XIII) and a compound of formula (XII) may conveniently be carried out in the presence of a catalyst such as tetrakis(triphenylphosphine)palladium(0), palladium(II) chloride or dichlorobis(triphenylphosphine)palladium(II), in the presence of a solvent such as tetrahydrofuran, 1,4-dioxane, 1,2-dimethoxyethane, toluene, methanol, or water, in the presence of a base such as sodium carbonate or potassium carbonate, and at a temperature in the range 10 to 250° C., preferably in the range 60 to 120° C.

Compounds of formula (VIII) may be prepared by reacting a compound of formula (XIII) with a compound of formula (VII), optionally in the presence of a suitable coupling reagent, such as bromo-tris-pyrrolidino-phosphonium hexafluorophosphate (PyBroP) or dicyclohexylcarbodiimide and 1-hydroxybenzotriazole, in the presence of a base such as triethylamine, N-methylmorpholine, diisopropylethylamine or potassium carbonate, in a solvent such as dichloromethane, N-methylpyrrolidinone, N,N-dimethylformamide or tetrahydrofuran, and at a temperature in the range from 0° C. to 150° C., in particular 50° C. to 100° C.

Alternatively, compounds of formula (VIII) may be prepared by reacting a compound of formula (XIII), with a compound of formula (X), and subsequently reacting the product of (X) and (XIII) with a compound of formula (V). The reaction of (X) and (XIII) may be conducted in the presence of a base such as triethylamine, N-methylmorpholine, diisopropylethylamine or potassium carbonate, and optionally in the presence of a suitable coupling reagent, such as bromo-tris-pyrrolidino-phosphonium hexafluorophosphate (PyBroP) or dicyclohexylcarbodiimide and 1-hydroxybenzotriazole. This reaction may conveniently be conducted in a solvent such as dichloromethane, N-methylpyrrolidinone, N,N-dimethylformamide or tetrahydrofuran, and at a temperature in the range from 0° C. to 150° C. The subsequent reaction with (V) may be conducted in an organic solvent such as N-methylpyrrolidinone, acetonitrile or N,N-dimethylformamide, optionally in the presence of a base such as potassium carbonate or triethylamine, and at a temperature in the range from 25° C. to 180° C., either in a microwave or under conventional thermal conditions.

Compounds of formula (XI) may be prepared by reacting a compound of formula (XIII), with a compound of formula (X), in the presence of a base such as triethylamine, N-methylmorpholine, diisopropylethylamine or potassium carbonate, and optionally in the presence of a suitable coupling reagent, such as bromo-tris-pyrrolidino-phosphonium hexafluorophosphate (PyBroP) or dicyclohexylcarbodiimide and 1-hydroxybenzotriazole. This reaction may conveniently be conducted in a solvent such as dichloromethane, N-methylpyrrolidinone, N,N-dimethylformamide or tetrahydrofuran, and at a temperature in the range from 0° C. to 150° C.

Compounds of formula (V), (VII), (IX), (X), (XII) and (XIII) are either commercially available, are known in the literature or may be prepared easily using known techniques.

It will be appreciated by those skilled in the art that in the processes of the present invention certain functional groups such as hydroxy, carboxyl or amino groups in the starting reagents or intermediate compounds may need to be protected by protecting groups. Thus, the preparation of the compounds of formula (I) may involve at a certain stage protection with and/or the removal of one or more protecting groups. The protection and deprotection of functional groups is described in ‘Protective Groups in Organic Synthesis’, 2nd edition, T. W. Greene and P. G. M. Wuts, Wiley-Interscience (1991) and ‘Protecting Groups’, P. J. Kocienski, Georg Thieme Verlag (1994). The compounds of formula (I) above may be converted to a pharmaceutically acceptable salt using conventional methods.

A compound of the invention, or a pharmaceutically acceptable salt thereof, can be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;

2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;

3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);

4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;

5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;

6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);

7. abdominal: hepatitis, including autoimmune, alcoholic and viral; fibrosis and cirrhosis of the liver; cholecystitis; pancreatitis, both acute and chronic;

8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);

9. allograft rejection: acute and chronic following, for example, transplantation of kidney, heart, liver, lung, bone marrow, skin or cornea or following blood transfusion; or chronic graft versus host disease;

10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;

11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;

12. other disorders with an inflammatory or immunological component; including acquired immune deficiency syndrome (AIDS), leprosy, Sezary syndrome, and paraneoplastic syndromes;

13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;

14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,

15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Accordingly, the present invention provides a compound of formula (IC), or a pharmaceutically acceptable salt thereof, for use in therapy,

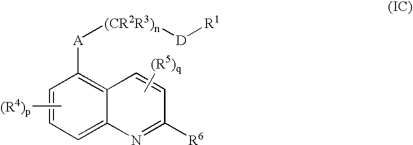

wherein

n is 1, 2 or 3;

when n is 1, A represents NHC(O) or NHC(S);

when n is 2 or 3, A represents NHC(O), C(O)NH, NHC(S) or C(S)NH;

D represents O, S or NR⁷, wherein R⁷ represents hydrogen, C₁₋₆ alkyl, C₁₋₆ hydroxyalkyl or C₁₋₆ haloalkyl;

R¹ represents a 6-10 membered aryl, or a 5-10 membered heteroaryl ring, which aryl or heteroaryl ring may be optionally substituted by one or more substituents independently selected from halogen, cyano, C₁₋₆ alkyl, C₁₋₆ hydroxyalkyl, C₁₋₆ haloalkyl, NR⁸R⁹, S(O)₀₋₂R¹⁰, S(O)₂NR¹¹R¹², C(O)NR¹³R¹⁴, CO₂R¹⁵, NR¹⁶S(O)₂R¹⁷, C(O)R¹⁸, NR¹⁹C(O)R²⁰, NR²¹C(O)NR²²R²³, NR²⁴S(O)₂NR²⁵R²⁶ and OR²⁷;

within each grouping CR²R³, R² and R³ each independently represent hydrogen, halogen, C₁₋₃ alkyl, C₁₋₃ hydroxyalkyl or C₁₋₃ haloalkyl;

p represents 0, 1, 2 or 3; each R⁴ independently represents halogen, cyano, C₁₋₆ alkyl, C₁₋₆ hydroxyalkyl or C₁₋₆ haloalkyl;

q represents 0, 1 or 2; each R⁵ independently represents halogen, cyano, C₁₋₆ alkyl, C₁₋₆ hydroxyalkyl or C₁₋₆ haloalkyl;

R⁶ represents a group

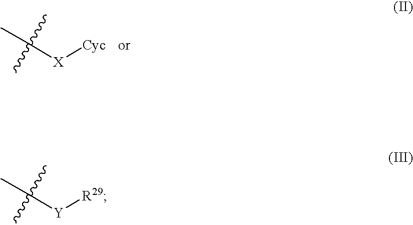

wherein X represents a bond, O, S, NR³⁰ or a C₁₋₄ alkylene which C₁₋₄ alkylene may be optionally substituted by one or more substituents independently selected from halogen, hydroxy and C₁₋₄alkoxy;

Cyc represents a 3-8 membered carbocyclic or a 4-8 membered heterocyclic ring, which ring may be optionally substituted by one or more substituents R²⁸, wherein each substituent R²⁸ is independently selected from halogen, cyano, ═O, S(O)₂R³¹, C(O)R³², CO₂R³³, C(O)NR³⁴R³⁵, NR³⁶C(O)R³⁷, S(O)₂NR³⁸R³⁹, NR⁴⁰S(O)₂R⁴¹, OR⁴², SR⁴³, NR⁴⁴R⁴⁵, Z¹ or a C₁₋₆ alkyl group which C₁₋₆ alkyl group may be optionally substituted by one or more substituents independently selected from halogen, hydroxyl, C₁₋₆ alkoxy, NR⁴⁶R⁴⁷, S(O)₀₋₂R⁴⁸, C(O)R⁴⁹, CO2R⁵⁰, C(O)NR⁵¹R⁵², NR⁵³C(O)R⁵⁴, S(O)₂NR⁵⁵R⁵⁶, NR⁵⁷S(O)₂R⁵⁸ and Z²;

Y represents a bond, OC₁₋₆ alkylene, S(O)₀₋₂C₁₋₆alkylene, N(R⁵⁹)C₁₋₆alkylene or C₁₋₆ alkylene, wherein any alkylene group in Y may be optionally substituted by one or more substituents independently selected from halogen, hydroxy and C₁₋₄ alkoxy;

R²⁹ represents hydrogen, halogen, hydroxyl, C₁₋₆alkoxy, cyano, NR⁶⁰R⁶¹, S(O)₀₋₂R⁶², C(O)R⁶³, CO₂R⁶⁴, C(O)NR⁶⁵R⁶⁶, NR⁶⁷C(O)R⁶⁸, S(O)₂NR⁶⁹R⁷⁰, NR⁷¹S(O)₂R⁷² or Z³;

with the proviso that when Y is a bond R²⁹ is not hydrogen;

R⁴², R⁴³, R⁴⁴ and R⁴⁵ each independently represent hydrogen or a C₁₋₆ alkyl group which C₁₋₆ alkyl group may be optionally substituted by one or more substituents independently selected from halogen, hydroxyl, C₁₋₆ alkoxy, NR⁷³R⁷⁴, S(O)₀₋₂R⁷⁵, C(O)R⁷⁶, CO₂R⁷⁷, C(O)NR⁷⁸R⁷⁹, NR⁸⁰C(O)R⁸¹, S(O)₂NR⁸²R⁸³, NR⁸⁴S(O)₂R⁸⁵ or Z⁴, or R⁴⁴ and R⁴⁵, together with the nitrogen atom to which they are both attached, may form a 4-8 membered aliphatic heterocyclic ring, which heterocyclic ring may be optionally substituted by one or more substituents independently selected from halogen, hydroxy, C₁₋₆ alkyl, C₁₋₆ hydroxyalkyl and C₁₋₆ haloalkyl;

Z¹, Z², Z³ and Z⁴ each independently represent tetrazole, or a 5-6 membered heterocyclic ring, which heterocyclic ring is substituted by one or more substituents independently selected from hydroxy, amino, ═O, ═S, and which heterocyclic ring may further be optionally substituted by one or more substituents independently selected from halogen and C₁₋₆ alkyl;

R⁸, R⁹, R¹¹, R¹², R¹³, R¹⁴, R¹⁵, R¹⁶, R¹⁸, R¹⁹, R²⁰, R²¹, R²², R²³, R²⁴, R²⁵, R²⁶, R²⁷, R³⁰, R³², R³³, R³⁴, R³⁵, R³⁶, R³⁷, R³⁸, R³⁹, R⁴⁰, R⁴⁶, R⁴⁷, R⁴⁹, R⁵⁰, R⁵¹, R⁵², R⁵³, R⁵⁴, R⁵⁵, R⁵⁶, R⁵⁷, R⁵⁹, R⁶⁰, R⁶¹, R⁶³, R⁶⁴, R⁶⁵, R⁶⁶, R⁶⁷, R⁶⁸, R⁶⁹, R⁷⁰, R⁷¹, R⁷³, R⁷⁴, R⁷⁶, R⁷⁷, R⁷⁸, R⁷⁹, R⁸⁰, R⁸¹, R⁸², R⁸³ and R⁸⁴ each independently represent hydrogen, C₁₋₆ alkyl, C₁₋₆ hydroxyalkyl or C₁₋₆ haloalkyl; or any of R⁸ and R⁹, R¹¹ and R¹², R¹³ and R¹⁴, R²² and R²³, R²⁵ and R²⁶, R³⁴ and R³⁵, R³⁸ and R³⁹, R⁴⁶ and R⁴⁷, R⁵¹ and R⁵², R⁵⁵ and R⁵⁶, R⁶⁰ and R⁶¹, R⁶⁵ and R⁶⁶, R⁶⁹ and R⁷⁰, R⁷³ and R⁷⁴, R⁷⁸ and R⁷⁹, and R⁸² and R⁸³, together with the nitrogen atom to which they are both attached, may form a 4-8 membered aliphatic heterocyclic ring, which heterocyclic ring may be optionally substituted by one or more substituents independently selected from halogen, hydroxy, C₁₋₆ alkyl, C₁₋₆ hydroxyalkyl and C₁₋₆ haloalkyl; and

R¹⁰, R¹⁷, R³¹, R⁴¹, R⁴⁸, R⁵⁸, R⁶², R⁷², R⁷⁵ and R⁸⁵ each independently represent C₁₋₆ alkyl, C₁₋₆ hydroxyalkyl or C₁₋₆ haloalkyl.

For compounds of formula (IC), embodiments of the invention include those wherein each of A, D, R¹, R², R³, R⁴, R⁵, R⁶, n, p and q are as defined herein above in embodiments of the invention concerning compounds of formula (I).

In another aspect, the invention provides the use of a compound of formula (IC), or a pharmaceutically acceptable salt thereof, as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

In another aspect, the invention provides the use of a compound of formula (IC), or a pharmaceutically acceptable thereof, as hereinbefore defined in the manufacture of a medicament for use in the treatment of rheumatoid arthritis.

In another aspect, the invention provides the use of a compound of formula (IC), or a pharmaceutically acceptable thereof, as hereinbefore defined, in the manufacture of a medicament for use in the treatment of osteoarthritis.

In another aspect, the invention provides the use of a compound of formula (IC), or a pharmaceutically acceptable thereof, as hereinbefore defined, in the manufacture of a medicament for use in the treatment of asthma or chronic obstructive pulmonary disease.

In another aspect, the invention provides the use of a compound of formula (IC), or a pharmaceutically acceptable thereof, as hereinbefore defined, in the manufacture of a medicament for use in the treatment of atherosclerosis.

In another aspect, the invention provides the use of a compound of formula (IC), or a pharmaceutically acceptable thereof, as hereinbefore defined, in the manufacture of a medicament for use in the treatment of inflammatory bowel disease.

The invention further provides a method of effecting immunosuppression (e.g. in the treatment of rheumatoid arthritis, osteoarthritis, irritable bowel disease, atherosclerosis or psoriasis) which comprises administering a therapeutically effective amount of a compound of formula (IC, or a pharmaceutically acceptable salt thereof, as hereinbefore defined to a patient.

The invention also provides a method of treating rheumatoid arthritis which comprises administering to a patient a therapeutically effective amount of a compound of formula (IC), or a pharmaceutically acceptable salt thereof, as hereinbefore defined to a patient.

The invention also provides a method of treating an obstructive airways disease (e.g. asthma or COPD) which comprises administering to a patient a therapeutically effective amount of a compound of formula (IC), or a pharmaceutically acceptable salt thereof, as hereinbefore defined to a patient.

In order to use a compound of the invention, or a pharmaceutically acceptable salt thereof, for the therapeutic treatment of a warm-blooded animal, such as man, said ingredient is normally formulated in accordance with standard pharmaceutical practice as a pharmaceutical composition.

Therefore in another aspect the present invention provides a pharmaceutical composition which comprises a compound of the formula (IC), or a pharmaceutically acceptable salt thereof (active ingredient), and a pharmaceutically acceptable adjuvant, diluent or carrier. In a further aspect the present invention provides a process for the preparation of said composition which comprises mixing active ingredient with a pharmaceutically acceptable adjuvant, diluent or carrier. Depending on the mode of administration, the pharmaceutical composition will, for example, comprise from 0.05 to 99% w (per cent by weight), such as from 0.05 to 80% w, for example from 0.10 to 70% w, such as from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 0.1 mg and 1 g of active ingredient.

In another aspect a pharmaceutical composition of the invention is one suitable for intravenous, subcutaneous or intramuscular injection. Each patient may receive, for example, an intravenous, subcutaneous or intramuscular dose of 0.01 mgkg⁻¹ to 100 mgkg⁻¹ of the compound, for example in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax I1-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY×1005.

The present invention further relates to the combination of a compound of the invention, or a pharmaceutically acceptable salt thereof, and a receptor antagonist for leukotrienes (LT) B4, LTC4, LTD4, and LTE4 selected from the group consisting of the phenothiazin-3-Is such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY×7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agents including muscarinic receptor (M1, M2, and M3) antagonist such as atropine, hyoscine, glycopyrrolate, ipratropium bromide, tiotropium bromide, oxitropium bromide, pirenzepine or telenzepine.

The present invention still further relates to the combination of a compound of the invention, or a pharmaceutically acceptable salt thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenytoin, sodium valproate, amitryptiline or other anti-depressant agent-s, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof.

A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B1.- or B2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK1 or NK3 receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), or (xxvi) inhibitor of transcription factor activation such as NFkB, API, or STATS.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);

(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;

(iii) an agent which inhibits cancer cell invasion (for example a metalloproteinase inhibitor like marimastat or an inhibitor of urokinase plasminogen activator receptor function);

(iv) an inhibitor of growth factor function, for example: a growth factor antibody (for example the anti-erbb2 antibody trastuzumab, or the anti-erbb 1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;

(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin (xvp3 function or an angiostatin);

(vi) a vascular damaging agent such as combretastatin A4, or a compound disclosed in WO 99/02166, WO 00/40529, WO 00/41669, WO 01/92224, WO 02/04434 or WO 02/08213;

(vii) an agent used in antisense therapy, for example one directed to one of the targets listed above, such as ISIS 2503, an anti-ras antisense;

(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or

(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be further explained by reference to the following illustrative examples. In the examples the NMR spectra were measured on a Varian Unity spectrometer at a proton frequency of either 300 or 400 MHz. The MS spectra were measured on either an Agilent 1100 MSD G1946D spectrometer or a Hewlett Packard HP1100 MSD G1946A spectrometer. Preparative HPLC separations were performed using a Waters Symmetry® or Xterra® column using 0.1% aqueous trifluoroacetic acid: acetonitrile, 0.1% aqueous ammonia: acetonitrile or 0.1% ammonium acetate: acetonitrile as the eluant. Microwave reactions were performed in a CEM Discover single mode microwave.

### EXAMPLE 1

### N-[6-Chloro-2-[(3R)-3-hydroxy-1-pyrrolidinyl]-5-quinolinyl]-2-(phenylamino)-acetamide

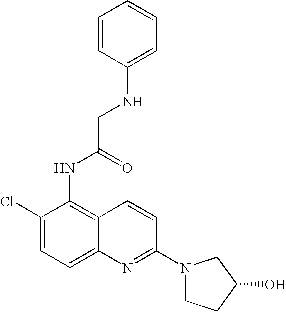

### a) (3R)-1-(5-Amino-6-chloro-2-quinolinyl)-3-pyrrolidinol

A mixture of (3R)-3-pyrrolidinol (620 mg), 2,6-dichloro-5-aminoquinoline (WO2005009968) (500 mg) and triethylamine (0.66 mL) in acetonitrile (3 mL) was heated with stirring in a microwave at 120° C. for 30 minutes. The products were concentrated in vacuo and purified by chromatography (SiO₂, dichloromethane:methanol:7N NH₃ in methanol 97:3:0.2 as eluant) to yield the sub-title compound as a solid (490 mg).

¹H NMR (300 MHz, d₆-DMSO) δ 8.30 (1H, d), 7.26 (1H, d), 6.82-6.69 (2H, m), 5.78 (2H, s), 4.95 (1H, d), 4.44-4.36 (1H, m), 3.65-3.39 (4H, m), 2.10-1.97 (1H, m), 1.95-1.85 (1H, m).

### b) 6-Chloro-2-[(3R)-3-[[(1,1-dimethylethyl)dimethylsilyl]oxy]-1-pyrrolidinyl]-5-quinolinamine

Chloro(1,1-dimethylethyl)dimethyl-silane (2.5 g) was added with stirring to a mixture of (3R)-1-(5-amino-6-chloro-2-quinolinyl)-3-pyrrolidinol (Example 1 (a)) (2.9 g) and imidazole (1.1 g) in N,N-dimethylformamide (50 mL). The mixture was stirred under nitrogen at room temperature for 24 hours and then water (100 mL) was added, the mixture was stirred for 5 minutes and the resulting precipitate was collected by filtration, washed with water (2×100 mL) and isohexane (2×100 mL), and dried to yield the sub-title compound as a solid (3.8 g).

¹H NMR (300 MHz, d₆-DMSO) δ 8.32 (1H, d), 7.27 (1H, d), 6.83-6.74 (2H, m), 5.80 (2H, s), 4.58 (1H, s), 3.72-3.49 (3H, m), 3.43-3.26 (1H, m), 2.16-2.02 (1H, m), 1.96-1.84 (1H, m), 0.85 (9H, s), 0.10 (6H, d).

### c) 2-Chloro-N-[6-chloro-2-[(3R)-3-[[(1,1-dimethylethyl)dimethylsilyl]oxy]-1-pyrrolidinyl]-5-quinolinyl]-acetamide

Chloroacetyl chloride (1.5 mL) was added dropwise to a stirred mixture of 6-chloro-2-[(3R)-3-[[(1,1-dimethylethyl)dimethylsilyl]oxy]-1-pyrrolidinyl]-5-quinolinamine (Example 1 (b)) (3.8 g) and potassium carbonate (4.1 g) in acetone (40 mL) under nitrogen at 0° C. The mixture was allowed to warm to room temperature and stirred for 2 hours. Potassium carbonate (2.0 g) and chloroacetyl chloride (0.75 mL) were added and the mixture was stirred for 2 hours. Water was added, the mixture was stirred for 10 minutes and the resulting precipitate was collected by filtration and purified by chromatography (SiO₂, isohexane:ethyl acetate 85:15-75:25 as eluant) to yield the sub-title compound as a solid (3.5 g).

¹H NMR (300 MHz, d₆-DMSO) δ 10.23 (1H, s), 7.92 (1H, d), 7.58 (1H, d), 7.51 (1H, d), 6.97 (1H, d), 4.64-4.55 (1H, m), 4.42 (2H, s), 3.76-3.53 (3H, m), 3.46-3.37 (1H, m), 2.20-2.05 (1H, m), 1.98-1.85 (1H, m), 0.86 (9H, s), 0.10 (6H, d).

### d) N-[6-Chloro-2-[(3R)-3-hydroxy-1-pyrrolidinyl]-5-quinolinyl]-2-(phenylamino)-acetamide

A mixture of 2-chloro-N-[6-chloro-2-[(3R)-3-[[(1,1-dimethylethyl)dimethylsilyl]oxy]-1-pyrrolidinyl]-5-quinolinyl]-acetamide (Example 1 (c)) (100 mg) and aniline (0.2 mL) were heated for 5 minutes with stirring at 120° C. in a microwave. The resulting products were purified by chromatography (SiO₂, ethyl acetate:isohexane:7N NH₃ in methanol 50:50:0.5 as eluant) to leave a solid which was dissolved in tetrahydrofuran (0.8 mL) and tetrabutylammonium fluoride (1M in tetrahydrofuran, 0.19 mL) was added. The mixture was stirred at room temperature for 3 hours, concentrated in vacuo and purified by chromatography (SiO₂, dichloromethane:methanol:7N NH₃ in methanol 95:5:0.5 as eluant) to give the title compound as a solid (35 mg).

MS: APCI(+ve) 397 (M+H⁺).

m.p. 230-232° C.

¹H NMR (300 MHz, d₆-DMSO) δ 9.91 (1H, s), 7.82 (1H, d), 7.54 (1H, d), 7.47 (1H, d), 7.15 (2H, dd), 6.86 (1H, d), 6.70 (2H, d), 6.63 (1H, t), 6.13 (1H, t), 4.98 (1H, d), 4.41 (1H, s), 3.98 (2H, d), 3.69-3.39 (4H, m), 2.11-1.86 (2H, m).

### EXAMPLE 2

### N-[6-Chloro-2-[(3R)-3-hydroxy-1-pyrrolidinyl]-5-quinolinyl]-2-[(3-chlorophenyl)amino]-acetamide monohydrochloride

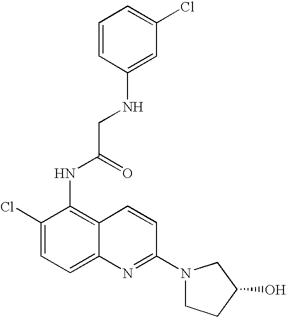

2-Chloro-N-[6-chloro-2-[(3R)-3-[[(1,1-dimethylethyl)dimethylsilyl]oxy]-1-pyrrolidinyl]-5-quinolinyl]-acetamide (Example 1 (c)) (150 mg) and 3-chloroaniline (0.35 mL) were heated at 140° C. for 30 minutes in a microwave. The resulting products were purified by chromatography (SiO₂, dichloromethane:methanol: 7N NH₃ in methanol 99:0.5:0.5 as eluant) to leave a solid which was dissolved in dichloromethane (0.5 mL) and hydrogen chloride (2M in diethyl ether, 0.1 mL) was added. The mixture was stirred at room temperature for 10 minutes and concentrated in vacuo to give the title compound as a solid (35 mg).

MS: APCI(+ve) 431 (M+H⁺).

m.p. 180° C. dec.

¹H NMR (300 MHz, d₆-DMSO) δ 10.39 (1H, s), 8.26-8.13 (2H, m), 7.91 (1H, d), 7.30 (1H, d), 7.14 (1H, t), 6.71 (1H, t), 6.68-6.60 (2H, m), 4.61-4.46 (1H, m), 4.08 (2H, s), 4.00-3.64 (4H, m), 2.21-1.97 (2H, m).

### EXAMPLE 3

### N-[6-Chloro-2-[(3R)-3-hydroxy-1-pyrrolidinyl]-5-quinolinyl]-2-[(4-chlorophenyl)amino]-acetamide monohydrochloride

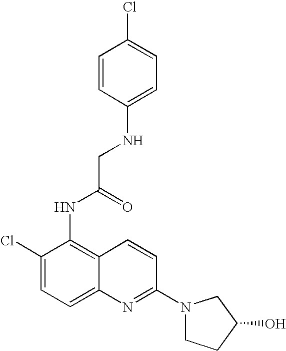

2-Chloro-N-[6-chloro-2-[(3R)-3-[[(1,1-dimethylethyl)dimethylsilyl]oxy]-1-pyrrolidinyl]-5-quinolinyl]-acetamide (Example 1 (c)) (150 mg) and 4-chloroaniline (211 mg) were heated at 130° C. for 15 minutes in a microwave. The resulting products were purified by chromatography (SiO₂, dichloromethane:methanol:7N NH₃ in methanol 98.5:1.5:0.5 as eluant) to leave a solid which was dissolved in dichloromethane (0.5 mL) and hydrogen chloride (2M in diethyl ether, 0.16 mL) was added. The mixture was stirred at room temperature for 15 minutes and concentrated in vacuo to give the title compound as a solid (40 mg).

MS: APCI(+ve) 431 (M+H⁺).

m.p. 175° C. dec.

¹H NMR (300 MHz, d₆-DMSO) δ 10.37 (1H, s), 8.23 (1H, d), 8.15 (1H, d), 7.91 (1H, d), 7.32 (1H, d), 7.18 (2H, d), 6.71 (2H, d), 4.60-4.46 (1H, m), 4.06 (2H, s), 3.99-3.62 (4H, m), 2.22-1.95 (2H, m).

### EXAMPLE 4

### N-[6-Chloro-2-[(3R)-3-hydroxy-1-pyrrolidinyl]-5-quinolinyl]-2-[(2-chlorophenyl)amino]-acetamide

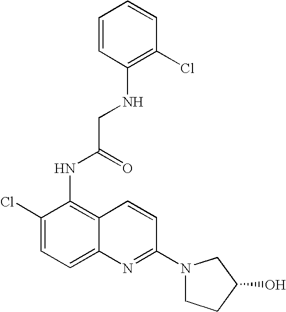

A mixture of 2-chloro-N-[6-chloro-2-[(3R)-3-[[(1,1-dimethylethyl)dimethylsilyl]oxy]-1-pyrrolidinyl]-5-quinolinyl]-acetamide (Example 1 (c)) (120 mg) and 2-chloroaniline (0.28 mL) were heated at 140° C. for 30 minutes in a microwave. The resulting products were purified by chromatography (SiO₂, ethyl acetate:isohexane 20:80, then ethyl acetate:isohexane:7N NH₃ in methanol 30:70:0.5 as eluant) to leave a solid which was dissolved in tetrahydrofuran (0.8 mL) and tetrabutylammonium fluoride (1M in tetrahydrofuran, 0.22 mL) was added. The mixture was stirred at room temperature for 3 hours, concentrated in vacuo and purified by chromatography (SiO₂, dichloromethane:methanol:7N NH₃ in methanol 99.5 :0.5:0.5-98.5:1.5:0.5 as eluant) to give the title compound as a solid (20 mg).

MS: APCI(+ve) 431 (M+H⁺).

m.p. 215-219° C.

¹H NMR (300 MHz, d₆-DMSO) δ 10.03 (1H, s), 7.88 (1H, d), 7.55 (1H, d), 7.48 (1H, d), 7.30 (1H, dd), 7.22 (1H, t), 6.89 (1H, d), 6.74 (1H, d), 6.67 (1H, dd), 5.86 (1H, t), 4.98 (1H, d), 4.41 (1H, s), 4.13 (2H, d), 3.70-3.39 (4H, m), 2.14-1.83 (2H, m).

### EXAMPLE 5

### N-[6-Chloro-2-[(3R)-3-hydroxy-1-pyrrolidinyl]-5-quinolinyl]-2-phenoxy-acetamide

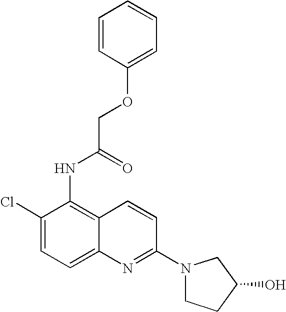

A mixture of 2-chloro-N-[6-chloro-2-[(3R)-3-[[(1,1-dimethylethyl)dimethylsilyl]oxy]-1-pyrrolidinyl]-5-quinolinyl]-acetamide (Example 1 (c)) (150 mg), phenol (310 mg), potassium carbonate (136 mg) and N-methylpyrrolidinone (0.75 mL) was heated at 100° C. for 10 minutes in a microwave. Water (20 mL) and dichloromethane (20 mL) were added, the layers separated, and the aqueous fraction was extracted with dichloromethane (20 mL). The combined organic layers were concentrated in vacuo and the residue was purified by chromatography (SiO₂, ethyl acetate:isohexane 20:80 as eluant) to leave a solid which was dissolved in tetrahydrofuran (0.8 mL) and tetrabutylammonium fluoride (1M in tetrahydrofuran, 0.3 mL) was added. The mixture was stirred at room temperature for 18 hours, concentrated in vacuo and purified by chromatography (SiO₂, dichloromethane:methanol 97.5:2.5) to give the title compound as a solid (40 mg).

MS: APCI(+ve) 398 (M+H⁺).

m.p. 233-235° C.

¹H NMR (300 MHz, d₆-DMSO) δ 10.09 (1H, s), 7.87 (1H, d), 7.57 (1H, d), 7.50 (1H, d), 7.40-7.31 (2H, m), 7.09 (2H, dd), 7.01 (1H, t), 6.90 (1H, d), 4.99 (1H, d), 4.84 (2H, s), 4.41 (1H, s), 3.71-3.40 (4H, m), 2.12-1.87 (2H, m).

### EXAMPLE 6

### N-[6-Chloro-2-[(3R)-3-hydroxy-1-pyrrolidinyl]-5-quinolinyl]-2-(phenylthio)-acetamide

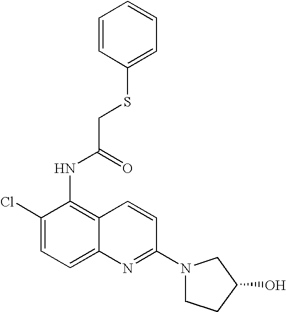

### a) N-[6-Chloro-2-[(3R)-3-[[(1,1-dimethylethyl)dimethylsilyl]oxy]-1-pyrrolidinyl]-5-quinolinyl]-2-(phenylthio)-acetamide

A mixture of 2-chloro-N-[6-chloro-2-[(3R)-3-[[(1,1-dimethylethyl)dimethylsilyl]oxy]-1-pyrrolidinyl]-5-quinolinyl]-acetamide (Example 1 (c)) (400 mg), benzenethiol (0.18 mL), triethylamine (0.37 mL) and acetonitrile (4 mL) was heated at 100° C. for 5 minutes in a microwave. The products were concentrated in vacuo and the residue was purified by chromatography (SiO₂, ethyl acetate:isohexane 20:80 as eluant) to give the sub-title compound as a solid (440 mg).

¹H NMR (300 MHz, d₆-DMSO) δ 10.11 (1H, s), 7.70 (1H, d), 7.58-7.44 (4H, m), 7.37 (2H, t), 7.25 (1H, t), 6.86 (1H, d), 4.59 (1H, s), 4.01 (2H, s), 3.74-3.51 (3H, m), 3.40 (1H, d), 2.19-2.04 (1H, m), 1.97-1.85 (1H, m), 0.86 (9H, s), 0.10 (6H, d).

### b) N-[6-Chloro-2-[(3R)-3-hydroxy-1-pyrrolidinyl]-5-quinolinyl]-2-(phenylthio)-acetamide

N-[6-Chloro-2-[(3R)-3-[[(1,1-dimethylethyl)dimethylsilyl]oxy]-1-pyrrolidinyl]-5-quinolinyl]-2-(phenylthio)-acetamide (Example 6 (a)) (40 mg) was dissolved in tetrahydrofuran (0.3 mL) and tetrabutylammonium fluoride (1M in tetrahydrofuran, 0.1 mL) was added. The mixture was stirred at room temperature for 4 hours, concentrated in vacuo and purified by chromatography (SiO₂, dichloromethane:methanol 98:2-95:5 as eluant) to give the title compound as a solid (18 mg).

MS: APCI(+ve) 414 (M+H⁺).

m.p. 246-249° C.

¹H NMR (300 MHz, d₆-DMSO) δ 10.11 (1H, s), 7.69 (1H, d), 7.58-7.44 (4H, m), 7.37 (2H, t), 7.25 (1H, t), 6.84 (1H, d), 4.98 (1H, d), 4.41 (1H, s), 4.01 (2H, s), 3.67-3.40 (4H, m), 2.13-1.85 (2H, m).

### EXAMPLE 7

### N-[6-Chloro-2-[(3R)-3-hydroxy-1-pyrrolidinyl]-5-quinolinyl]-2-[(3-cyanophenyl)amino]-acetamide

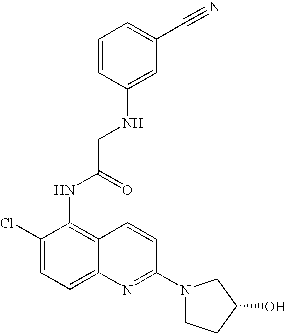

Prepared according to the method of Example 1 (d) using 2-chloro-N-[6-chloro-2-[(3R)-3-[[(1,1-dimethylethyl)dimethylsilyl]oxy]-1-pyrrolidinyl]-5-quinolinyl]-acetamide (Example 1 (c)) (120 mg) and 3-aminobenzonitrile (310 mg) heating at 120° C. for 10 minutes in a microwave, and then tetrabutylammonium fluoride (1M in tetrahydrofuran, 0.2 mL) and tetrahydrofuran (0.8 mL). Purification by chromatography (SiO₂, dichloromethane:methanol: 7N NH₃ in methanol 97.5:2.5:0.5-96:4:0.5 as eluant) gave the title compound as a solid (30 mg).

MS: APCI(+ve) 422 (M+H⁺).

m.p. 254-256° C.

¹H NMR (300 MHz, d₆-DMSO) δ 10.03 (1H, s), 7.88 (1H, d), 7.56 (1H, d), 7.48 (1H, d), 7.37-7.28 (1H, m), 7.05-6.98 (3H, m), 6.88 (1H, d), 6.72 (1H, t), 4.99 (1H, d), 4.41 (1H, s), 4.08 (2H, d), 3.69-3.40 (4H, m), 2.12-1.87 (2H, m).

### EXAMPLE 8

### N-[6-Chloro-2-[(3R)-3-hydroxy-1-pyrrolidinyl]-5-quinolinyl]-2-[(4-cyanophenyl)amino]-acetamide

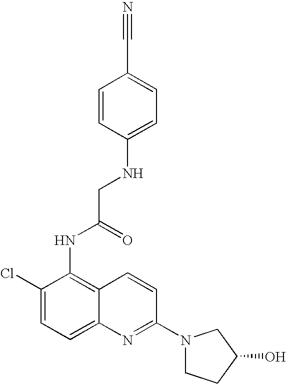

Prepared according to the method of Example 1 (d) using 2-chloro-N-[6-chloro-2-[(3R)-3-[[(1,1-dimethylethyl)dimethylsilyl]oxy]-1-pyrrolidinyl]-5-quinolinyl]-acetamide (Example 1 (c)) (120 mg) and 4-aminobenzonitrile (310 mg) heating at 110° C. for 45 minutes in a microwave, and then tetrabutylammonium fluoride (1M in tetrahydrofuran, 0.2 mL) and tetrahydrofuran (0.8 mL). Purification by chromatography (SiO₂, dichloromethane:methanol: 7N NH₃ in methanol 98:2:0.5-96:4:0.5 as eluant) gave the title compound as a solid (30 mg).

MS: APCI(+ve) 422 (M+H⁺).

m.p. 230-233° C.

¹H NMR (300 MHz, d₆-DMSO) δ 10.04 (1H, s), 7.88 (1H, d), 7.61-7.45 (4H, m), 7.14 (1H, t), 6.91 (1H, d), 6.77 (2H, d), 4.99 (1H, d), 4.42 (1H, s), 4.12 (2H, d), 3.71-3.41 (4H, m), 2.13-1.86 (2H, m).

### EXAMPLE 9

### N-[6-Chloro-2-[(3R)-3-hydroxy-1-pyrrolidinyl]-5-quinolinyl]-2-[(3-fluorophenyl)amino]-acetamide

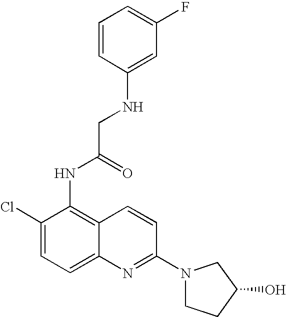

Prepared according to the method of Example 1 (d) using 2-chloro-N-[6-chloro-2-[(3R)-3-[[(1,1-dimethylethyl)dimethylsilyl]oxy]-1-pyrrolidinyl]-5-quinolinyl]-acetamide (Example 1 (c)) (120 mg) and 3-fluoroaniline (0.25 mL) heating at 120° C. for 15 minutes in a microwave, and then tetrabutylammonium fluoride (1M in tetrahydrofuran, 0.25 mL) and tetrahydrofuran (0.8 mL). Purification by chromatography (SiO₂, dichloromethane:methanol: 7N NH₃ in methanol 98:2:0.5-96:4:0.5 as eluant) gave the title compound as a solid (50 mg).

MS: APCI(+ve) 415 (M+H⁺).

m.p. 218-220° C.

¹H NMR (300 MHz, d₆-DMSO) δ 9.97 (1H, s), 7.85 (1H, d), 7.55 (1H, d), 7.48 (1H, d), 7.14 (1H, q), 6.87 (1H, d), 6.55-6.34 (4H, m), 4.98 (1H, d), 4.41 (1H, s), 4.02 (2H, d), 3.67-3.41 (4H, m), 2.12-1.86 (2H, m).

### EXAMPLE 10

### N-[6-Chloro-2-[(3R)-3-hydroxy-1-pyrrolidinyl]-5-quinolinyl]-2-[(4-fluorophenyl)amino]-acetamide

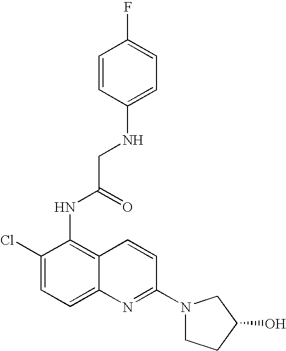

Prepared according to the method of Example 1 (d) using 2-chloro-N-[6-chloro-2-[(3R)-3-[[(1,1-dimethylethyl)dimethylsilyl]oxy]-1-pyrrolidinyl]-5-quinolinyl]-acetamide (Example 1 (c)) (120 mg) and 4-fluoroaniline (0.3 mL) heating at 120° C. for 5 minutes in a microwave, and then tetrabutylammonium fluoride (1M in tetrahydrofuran, 0.26 mL) and tetrahydrofuran (0.8 mL). Purification by chromatography (SiO₂, dichloromethane:methanol: 7N NH₃ in methanol 98:2:0.5-96:4:0.5 as eluant) gave the title compound as a solid (65 mg).

MS: APCI(+ve) 415 (M+H⁺).

m.p. 242-246° C.

¹H NMR (300 MHz, d₆-DMSO) δ 9.92 (1H, s), 7.82 (1H, d), 7.54 (1H, d), 7.47 (1H, d), 7.00 (2H, t), 6.87 (1H, d), 6.68 (2H, dd), 6.10 (1H, t), 4.98 (1H, d), 4.42 (1H, s), 3.97 (2H, d), 3.70-3.41 (4H, m), 2.13-1.85 (2H, m).

### EXAMPLE 11

### N-[6-Chloro-2-[(3R)-3-hydroxy-1-pyrrolidinyl]-5-quinolinyl]-2-[(3,4-difluorophenyl)amino]-acetamide

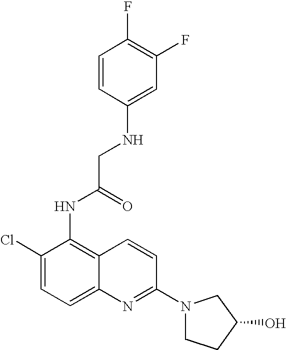

Prepared according to the method of Example 1 (d) using 2-chloro-N-[6-chloro-2-[(3R)-3-[[(1,1-dimethylethyl)dimethylsilyl]oxy]-1-pyrrolidinyl]-5-quinolinyl]-acetamide (Example 1 (c)) (105 mg) and 3,4-difluoroaniline (0.23 mL) heating at 120° C. for 10 minutes in a microwave, and then tetrabutylammonium fluoride (1M in tetrahydrofuran, 0.2 mL) and tetrahydrofuran (0.8 mL). Purification by chromatography (SiO₂, dichloromethane:methanol: 7N NH₃ in methanol 99:1:0.5-98:2:0.5 as eluant) gave the title compound as a solid (45 mg).

MS: APCI(+ve) 433 (M+H⁺).

m.p. 238-240° C.

¹H NMR (300 MHz, d₆-DMSO) δ 9.97 (1H, s), 7.86 (1H, d), 7.55 (1H, d), 7.48 (1H, d), 7.19 (1H, dd), 6.88 (1H, d), 6.65 (1H, ddd), 6.50-6.42 (1H, m), 6.37 (1H, t), 4.99 (1H, s), 4.42 (1H, s), 4.00 (2H, d), 3.69-3.41 (4H, m), 2.14-1.86 (2H, m).

### EXAMPLE 12

### N-[6-Chloro-2-[(3R)-3-hydroxy-1-pyrrolidinyl]-5-quinolinyl]-2-[(3,4-difluorophenyl)amino]-propanamide

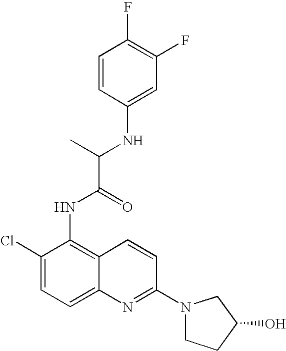

### a) 2-Chloro-N-[6-chloro-2-[(3R)-3-[[(1,1-dimethylethyl)dimethylsilyl]oxy]-1-pyrrolidinyl]-5-quinolinyl]-propanamide

2-Chloro-propanoyl chloride (0.15 mL) was added to a stirred solution of 6-chloro-2-[3-[[(1,1-dimethylethyl)dimethylsilyl]oxy]-1-pyrrolidinyl]-5-quinolinamine (Example 1 (b)) (520 mg) and triethylamine (0.42 mL) in dichloromethane (20 mL) at 0° C. under nitrogen. The mixture was allowed to warm to room temperature over 18 hours, dichloromethane and water were added and the layers were separated. The aqueous fraction was extracted with dichloromethane and the combined organic extracts were dried (MgSO₄), filtered and concentrated in vacuo. Purification by chromatography (SiO₂, isohexane:ethyl acetate 80:20 as eluant) gave the sub-title compound as a foam (350 mg).

¹H NMR (300 MHz, CDCl₃) δ 8.20 (1H, s), 7.77 (1H, d), 7.63 (1H, d), 7.52 (1H, d), 6.76 (1H, d), 4.69 (1H, q), 4.56 (1H, s), 3.72 (3H, m), 3.52 (1H, m), 2.08 (2H, m), 1.92 (3H, d), 0.89 (9H, s), 0.10 (3H, s), 0.10 (3H, s).

### b) N-[6-Chloro-2-[(3R)-3-hydroxy-1-pyrrolidinyl]-5-quinolinyl]-2-[(3,4-difluorophenyl)amino]-propanamide

Prepared according to the method of Example 1 (d) using 2-chloro-N-[6-chloro-2-[(3R)-3-[[(1,1-dimethylethyl)dimethylsilyl]oxy]-1-pyrrolidinyl]-5-quinolinyl]-propanamide (Example 12 (a)) (150 mg) and 3,4-difluoroaniline (500 mg) heating at 120° C. for 25 minutes in a microwave, and then tetrabutylammonium fluoride (1M in tetrahydrofuran, 1.0 mL) and tetrahydrofuran (5 mL). Purification by trituration with diethyl ether gave the title compound as a solid (82 mg).

MS: APCI(+ve) 447 (M+H⁺).

¹H NMR (400 MHz, CD₃OD) δ 7.65 (1H, d), 7.58 (1H, d), 7.51 (1H, d), 7.06 (1H, q), 6.80 (1H, d), 6.63 (1H, m), 6.50 (1H, m), 4.53 (1H, m), 4.05 (1H, q), 3.68 (3H, m), 3.57 (1H, br d), 2.14 (1H, m), 2.06 (1H, m), 1.66 (3H, d).

### EXAMPLE 13

### N-[6-Chloro-2-(1,2-dihydroxypropyl)-5-quinolinyl]-2-[(4-fluorophenyl)amino]-acetamide

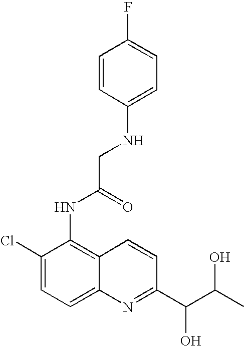

### a) (2,6-Dichloro-5-quinolinyl)-[(1,1-dimethylethoxy)carbonyl]-carbamic acid 1,1-dimethylethyl ester

Di-tert-butyl dicarbonate (3.3 g) was added to a solution of 2,6-dichloro-5-aminoquinoline (WO2005009968) (1.1 g) and ⁴-dimethylaminopyridine (20 mg) in acetonitrile (25 mL) at room temperature under nitrogen. The reaction was heated to 80° C. for 4 hours. The reaction was then concentrated in vacuo and the residue partitioned between diethyl ether and water. The phases were separated, the aqueous phase was further extracted with diethyl ether and the combined organic extracts were dried (Na₂SO₄), filtered and concentrated in vacuo. The residual solid was triturated with diethyl ether to give the sub-title compound as a pale brown solid (1.7 g).

¹H NMR (400 MHz, CDCl₃) δ 8.1 1(1H, d), 7.97 (1H, d), 7.77 (1H, d), 7.48 (1H, d), 1.33 (18H, s).

### b) 6-Chloro-2-(2-propenyl)-5-quinolinyl]-[(1,1-dimethylethoxy)carbonyl]-carbamic acid 1,1-dimethylethyl ester

A solution of (2,6-dichloro-5-quinolinyl)-[(1,1-dimethylethoxy)carbonyl]-carbamic acid 1,1-dimethylethyl ester (Example 13(a)) (1.7 g), 4,4,5,5-tetramethyl-2-(2-propenyl)-1,3,2-dioxaborolane (1.4 g), potassium carbonate (1.2 g) and tetrakis(triphenylphosphine) palladium(0) (490 mg) in tetrahydrofuran (10 mL) and water (10 mL) was heated in a sealed vessel at 60° C. for 4 hours. The reaction was concentrated in vacuo beore re-dissolving in dichloromethane and washing with water. The organic layer was separated, dried (Na₂SO₄), filtered and concentrated in vacuo. Purification by chromatography (SiO₂, iso-hexane:dichloromethane 50:50-10:90 as eluant) gave the sub-title compound as an oil (1.7 g).

¹H NMR (400 MHz, CDCl₃) δ 8.08 (1H, d), 7.98 (1H, d), 7.70 (1H, d), 7.40 (1H, d), 6.15 (1H, m), 5.21 (2H, m), 3.78 (2H, d), 1.31 (18H, s).

### c) 2-Chloro-N-[6-chloro-2-(1-propenyl)-5-quinolinyl]-acetamide

4N HCl in dioxane was added to 6-chloro-2-(2-propenyl)-5-quinolinyl]-[(1,1-dimethylethoxy)carbonyl]-carbamic acid 1,1-dimethylethyl ester (Example 13 (b)) (1.7 g) and the mixture was stirred under nitrogen for 40 minutes, after which time the reaction was concentrated in vacuo. The residue was then redissolved in acetone (40 mL), treated with potassium carbonate (2.3 g) and chloroacetyl chloride (0.61 mL). The reaction was stirred for 18 hours under nitrogen before being concentrated in vacuo and partitioned between water and ethyl acetate. The phases were separated and the water layer was further extracted with ethyl acetate. The combined organic extracts were dried (Na₂SO₄), filtered and concentrated in vacuo. Purification by chromatography (SiO₂, dichloromethane-dichloromethane:acetonitrile 20:1 as eluant) gave the sub-title compound as a solid (350 mg).

¹H NMR (400 MHz, DMSO) δ 8.21 (1H, d), 7.90 (1H, d), 7.82 (1H, d), 7.78 (1H, d), 6.97 (1H, m), 6.71 (1H, d), 4.45 (2H, s), 1.98 (3H, d).

### d) 2-Chloro-N-[6-chloro-2-(1,2-dihydroxypropyl)-5-quinolinyl]-acetamide

A solution of 2-chloro-N-[6-chloro-2-(1-propenyl)-5-quinolinyl]-acetamide (Example 13(c)) (350 mg) in acetone (3 mL) and water (3 mL) was treated with 4-methylmorpholine N-oxide (318 mg) and potassium osmate (20 mg). The reaction was then stirred under nitrogen at room temperature for 18 hours. After this time a solution of saturated sodium metabisulphite was added and stirring continued for 30 minutes. Water was added and the mixture was extracted with dichloromethane and ethyl acetate. The combined organic extracts were dried (Na₂SO₄), filtered and concentrated in vacuo. Trituration with dichloromethane gave the sub-title compound as a colourless solid (120 mg).

¹H NMR (400 MHz, CD₃OD) δ 8.40 (1H, d), 8.10 (1H, d), 7.91 (1H, d), 7.85 (1H, d), 4.75 (1H, d), 4.47 (2H, s), 4.17 (1H, m), 1.24, (3H, d).

### e) N-[6-Chloro-2-(1,2-dihydroxypropyl)-5-quinolinyl]-2-[(4-fluorophenyl)amino]-acetamide

A solution of 2-chloro-N-[6-chloro-2-(1,2-dihydroxypropyl)-5-quinolinyl]-acetamide (Example 13(d)) (120 mg) in acetonitrile (2.5 mL) was treated with 4-fluoroaniline (200 mg) and diisopropylethylamine (0.3 mL). The reaction was heated in the microwave with stirring at 100° C. for 30 mins before being cooled to room temperature and concentrated in vacuo. Purification of the residue by trituration with iso-hexane and then by HPLC (Waters Symmetry column, aqueous ammonium acetate:acetonitrile) gave the title compound as a colourless solid (25 mg).

MS: APCI(+ve) 404 (M+H⁺).

m.p. 135-138° C.

¹H NMR (400 MHz, CD₃OD) δ 8.21 (1H, d), 8.06 (1H, d), 7.87 (1H, d), 7.76(1H, d), 7.04 (2H, t), 6.84 (2H, m), 4.73 (1H, d), 4.17-4.10 (3H, m), 1.23 (3H, d).

### EXAMPLE 14

### N-(6-Chloro-2-morpholin-4-yl-quinolin-5-yl)-2-(4-fluoro-phenylamino)-acetamide

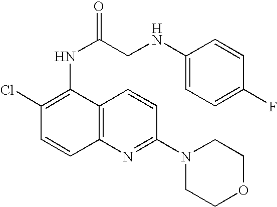

### a) (6-Chloro-2-morpholin-4-yl-quinolin-5-yl)-carbamic acid tert-butyl ester

A solution of (2,6-dichloro-5-quinolinyl)-[(1,1-dimethylethoxy)carbonyl]-carbamic acid 1,1-dimethylethyl ester (Example 13(a)) (2.07 g), morpholine (0.70 mL) and triethylamine (1.4 mL) were heated in a sealed vessel to 110° C. for 4 hours. The reaction was cooled to room temperature and concentrated in vacuo. The residue was partitioned between water and ethyl acetate, further extracting with ethyl acetate (2×100 mL). The combined organic extracts were washed with 10% K₂CO₃ (aq), dried (MgSO₄), filtered and concentrated in vacuo. Purification by chromatography (SiO₂, isohexane:ethyl acetate 80:20 as eluant) gave the sub-title compound as a solid (1.9 g).

¹H NMR (400 MHz, d₆-DMSO) δ 9.05 (1H, s), 7.96 (1H, d), 7.59 (1H, d), 7.49 (1H, d), 7.33 (1H, d), 3.72 (2H, m), 3.67 (2H, m), 3.52 (2H, m) 3.28 (2H, m), 1.40 (9H, s).

### b) 6-Chloro-2-morpholin-4-yl-quinolin-5-ylamine

(6-Chloro-2-morpholin-4-yl-quinolin-5-yl)-carbamic acid tert-butyl ester (Example 14 (a)) (2.2 g) in dichloromethane (30 mL) was treated with trifluoroacetic acid (10 mL) and the reaction was stirred at room temperature overnight. The reaction was concentrated in vacuo before dissolving in dichloromethane and washing with saturated sodium hydrogen carbonate. The organic layer was dried (MgSO₄), filtered and concentrated in vacuo to give the sub-title compound as a solid (1.0 g) which was used in the next step without further purification.

MS: APCI(+ve) 264 (M+H⁺).

### c) 2-Chloro-N-(6-chloro-2-morpholin-4-yl-quinolin-5-yl)-acetamide

6-Chloro-2-morpholin-4-yl-quinolin-5-ylamine (Example 14 (b)) (1.0 g) in acetone (15 mL) was treated with K₂CO₃ (1.57 g) and cooled to 0° C. before the addition of chloroacetyl chloride (0.45 mL). The reaction was stirred for 16 hours before being filtered, washing through with acetone. The filtrates were concentrated in vacuo and the residue triturated with diethyl ether to give a pale gray solid (0.38 g).

MS: APCI(−ve) 388 (M−H⁺).

### d) N-(6-Chloro-2-morpholin-4-yl-quinolin-5-yl)-2-(4-fluoro-phenylamino)-acetamide

2-Chloro-N-(6-chloro-2-morpholin-4-yl-quinolin-5-yl)-acetamide (Example 14 (c)) (0.2 g) in acetonitrile (4 mL) was treated with 4-fluoroaniline (0.33 g) and diisopropylethylamine (0.53 mL) before being heated to 110° C. for 2 hours. The reaction was cooled to room temperature, concentrated in vacuo and triturated with iso-Hexane. The residue was then redissolved in acetonitrile methanol mixture and purified by HPLC (Xterra column, aqueous ammonium acetate:acetonitrile) gave the title compound as a colourless solid (30 mg).

MS: APCI(+ve) 415 (M+H⁺).

¹H NMR (400 MHz, d₆-DMSO) δ 9.95 (1H, s), 7.92 (1H, d), 7.59 (1H, d), 7.51 (1H, d), 7.25 (1H, d), 6.99 (2H, m), 6.67 (2H, m), 6.11 (H, q), 3.97 (2H, d), 3.72-3.65 (8H, complex)

### EXAMPLE 15

### N-[6-Chloro-2-(1H-pyrazol-3-yl)-quinolin-5-yl]-2-(4-fluoro-phenylamino)-acetamide

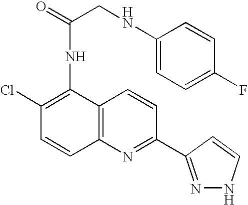

### a) [6-Chloro-2-(1H-pyrazol-3-yl)-quinolin-5-yl]-carbamic acid tert-butyl ester

A solution of (2,6-dichloro-5-quinolinyl)-[(1,1-dimethylethoxy)carbonyl]-carbamic acid 1,1-dimethylethyl ester (Example 13(a)) (1.65 g), 1H-pyrazole-5-boronic acid (0.9 g) cesium carbonate (2.6 g) and tetrakis(triphenylphosphine)palladium(0) (1.2 g) were combined and suspended in dioxane (12 mL) and water (5 mL). The reaction was heated overnight at reflux before being concentrated in vacuo. The residue was partitioned between water and dichloromethane, further extracting with dichloromethane (2×50 mL). The combined organic extracts were dried (MgSO₄), filtered and concentrated in vacuo to give the sub-title compound as a solid (1.0 g). Progressed to next stage without further purification.

MS: APCI(−ve) 343 (M−H⁺).

### b) 6-chloro-2-(1H-pyrazol-3-yl)quinolin-5-amine

[6-Chloro-2-(1H-pyrazol-3-yl)-quinolin-5-yl]-carbamic acid tert-butyl ester (Example 15(a)) (1.0 g), was dissolved in 4N HCl in Dioxane (30 mL) and stirred for 2 hours. The reaction was concentrated in vacuo, partitioned between water and dichloromethane, further extracting with dichloromethane (2×50 mL). The combined organic extracts were dried (MgSO₄), filtered and concentrated in vacuo. Purification by chromatography (SiO₂, isohexane:ethyl acetate 1:1 as eluant) gave the sub-title compound as a solid (0.7 g).

MS: APCI(−ve) 245.5 (M−H⁺).

### c) 6-Chloro-2-[1-(2-trimethylsilanyl-ethoxymethyl)-1H-pyrazol-3-yl]-quinolin-5-ylamine

6-chloro-2-(1H-pyrazol-3-yl)quinolin-5-amine (Example 15(b)) (0.7 g) was dissolved in dimethylformamide (10 mL) cooled to −10° C. and treated with sodium hydride (200 mg). The reaction was stirred for 20 minutes before 2-(trimethylsilyl)ethoxymethyl chloride (0.89 mL) was added and stirring continued for 18 hours at room temperature. The reaction was partitioned between water and ethyl acetate, further washing the organic layer with water (3×50 mL). The organic layer was washed with saturated sodium chloride solution, dried (MgSO₄), filtered and concentrated in vacuo. Purification by chromatography (SiO₂, isohexane:ethyl acetate 80:20 as eluant) gave the crude sub-title compound as a yellow oil (0.4 g) which was used without further purification.

MS: APCI(+ve) 375 (M+H⁺).

### d) 2-Chloro-N-{6-chloro-2-[1-(2-trimethylsilanyi-ethoxymethyl)-1H-pyrazol-3-yl]-quinolin-5-yl}-acetamide

Chloroacetyl chloride (0.17 mL) was added dropwise to a stirred mixture of 6-Chloro-2-[1-(2-trimethylsilanyl-ethoxymethyl)-1H-pyrazol-3-yl]-quinolin-5-ylamine (Example 15 (c)) (0.4 g) and potassium carbonate (0.44 g) in acetone (10 mL) under nitrogen at 0° C. The reaction was concentrated in vacuo, partitioned between water and ethyl acetate, further extracting with ethyl acetate (2×50 mL). The combined organic extracts were dried (MgSO₄), filtered and concentrated in vacuo. Purification by chromatography (SiO₂, isohexane:ethyl acetate 70:30 as eluant) gave the sub-title compound as a yellow is 5 oil (0.16 g).

MS: APCI(−ve) 449 (M−H⁺).

### e) N-{6-Chloro-2-[1-(2-trimethylsilanyl-ethoxymethyl)-1H-pyrazol-3-yl]-quinolin-5-yl}-2-(4-fluoro-phenylamino)-acetamide

2-Chloro-N-{6-chloro-2-[1-(2-trimethylsilanyl-ethoxymethyl)-1H-pyrazol-3-yl]-quinolin-5-yl}-acetamide (Example 15 (d)) (0.16 g) was dissolved in acetonitrile (2 mL). The solution was treated with 4-fluoroaniline (0.2 g) and triethylamine (0.24 mL) before being heated to 100° C. for 30 minutes. The reaction was cooled to room temperature, concentrated in vacuo and triturated with iso-Hexane. The residue was then redissolved in acetonitrile methanol mixture and purified by HPLC (Xterra column, aqueous ammonium acetate:acetonitrile) to give the title compound as a white solid (50 mg).

MS: APCI(−ve) 524 (M−H⁺).

### f) N-[6-Chloro-2-(1H-pyrazol-3-yl)-quinolin-5-yl]-2-(4-fluoro-phenylamino)-acetamide

N-{6-Chloro-2-[1-(2-trimethylsilanyl-ethoxymethyl)-1 H-pyrazol-3-yl]-quinolin-5-yl}-2-(4-fluoro-phenylamino)-acetamide (Example 15 (e)) (40 mg) was dissolved in 4N HCl in Dioxane and stirred for 48 hours. The reaction was concentrated in vacuo, redissolved in methanol and purified by HPLC (Xterra column, aqueous ammonium acetate:acetonitrile) to give the title compound as a white solid (10 mg).

MS: APCI(−ve) 394 (M−H⁺).

¹H NMR (400 MHz, CD₃OD) δ 8.17 (2H, d), 8.04 (1H, d), 7.79 (1H, d), 7.76 (1H, broad s), 7.10 (1H, broad s), 6.97 (2H, t), 6.77 (2H, m), 4.07 (2H, s)

Pharmacological Analysis

Certain compounds such as benzoylbenzoyl adenosine triphosphate (bbATP) are known to be agonists of the P2X₇ receptor, effecting the formation of pores in the plasma membrane (Drug Development Research (1996), 37(3), p. 126). Consequently, when the receptor is activated using bbATP in the presence of ethidium bromide (a fluorescent DNA probe), an increase in the fluorescence of intracellular DNA-bound ethidium bromide is observed. The increase in fluorescence can be used as a measure of P2X₇ receptor activation and therefore to quantify the effect of a compound on the P2X₇ receptor.

In this manner, each of the title compounds of the Examples was tested for antagonist activity at the P2X₇ receptor. Thus, the test was performed in 96-well flat bottomed microtitre plates, the wells being filled with 250 μl of test solution comprising 200 μl of a suspension of THP-1 cells (2.5×10⁶ cells/ml) containing 10⁻⁴M ethidium bromide, 25 μl of a high potassium buffer solution containing 10⁻⁵M bbATP, and 25 μl of the high potassium buffer solution containing concentrations of test compound typically from 30 μM-0.001 μM. The plate was covered with a plastics sheet and incubated at 37° C. for one hour. The plate was then read in a Perkin-Elmer fluorescent plate reader, excitation 520 nm, emission 595 nm, slit widths: Ex 15 nm, Em 20 nm. For the purposes of comparison, bbATP (a P2X₇ receptor agonist) and pyridoxal 5-phosphate (a P2X₇ receptor antagonist) were used separately in the test as controls. From the readings obtained, a pIC₅₀ figure was calculated for each test compound, this figure being the negative logarithm of the concentration of test compound necessary to reduce the bbATP agonist activity by 50%. Each of the compounds of the Examples demonstrated antagonist activity, having a pIC₅₀ figure >5.5. For example, the following table shows the pIC₅₀ figures for a representative selection of compounds:

| Compound of | |
|---|---|
| Example No. | pIC₅₀ |
| 6 | 6.4 |
| 7 | 7.1 |

What is claimed is:

1. A compound of formula (I), or a pharmaceutically acceptable salt thereof, 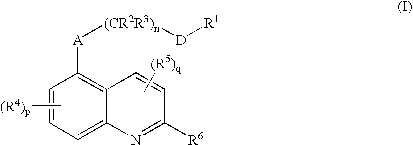
wherein
n is 1, 2 or 3;
when n is 1, A represents NHC(O) or NHC(S);
when n is 2 or 3, A represents NHC(O), C(O)NH, NHC(S) or C(S)NH;
D represents O, S or NR⁷, wherein R⁷ represents hydrogen, C₁₋₆ alkyl, C₁₋₆ hydroxyalkyl or C₁₋₆ haloalkyl;
R¹ represents a 6-10 membered aryl, or a 5-10 membered heteroaryl ring, which aryl or heteroaryl ring may be optionally substituted by one or more substituents independently selected from halogen, cyano, C₁₋₆ alkyl, C₁₋₆ hydroxyalkyl, C₁₋₆ haloalkyl, NR⁸R⁹, S(O)₀₋₂R¹⁰, S(O)₂NR¹¹R¹², C(O)NR¹³R¹⁴, CO₂R¹⁵, NR¹⁶S(O)₂R¹⁷, C(O)R¹⁸, NR¹⁹C(O)R²⁰, NR²¹C(O)NR²²R²³, NR²⁴S(O)₂NR²⁵R²⁶ and OR²⁷;
within each grouping CR²R³, R² and R³ each independently represent hydrogen, halogen, C₁₋₃ alkyl, C₁₋₃ hydroxyalkyl or C₁₋₃ haloalkyl;
p represents 0, 1, 2 or 3; each R⁴ independently represents halogen, cyano, C₁₋₆ alkyl, C₁₋₆ hydroxyalkyl or C₁₋₆ haloalkyl;
q represents 0, 1 or 2; each R⁵ independently represents halogen, cyano, C₁₋₆ alkyl, C₁₋₆ hydroxyalkyl or C₁₋₆ haloalkyl;
R⁶ represents a group 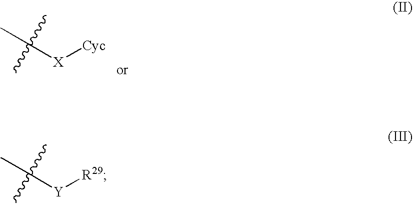
wherein X represents a bond, O, S, NR³⁰ or a C₁₋₄ alkylene which C₁₋₄ alkylene may be optionally substituted by one or more substituents independently selected from halogen, hydroxy and C₁₋₄alkoxy;
Cyc represents a 3-8 membered carbocyclic or a 4-8 membered heterocyclic ring, which ring may be optionally substituted by one or more substituents R²⁸, wherein each substituent R²⁸ is independently selected from halogen, cyano, ═O, S(O)₂R³¹, C(O)R³², CO₂R³³, C(O)NR³⁴R³⁵, NR³⁶C(O)R³⁷, S(O)₂NR³⁸R³⁹, NR⁴⁰S(O)₂R⁴¹, OR⁴², SR⁴³, NR⁴⁴R⁴⁵, Z¹ or a C₁₋₆ alkyl group which C₁₋₆ alkyl group may be optionally be substituted by one or more substituents independently selected from halogen, hydroxyl, C₁₋₆ alkoxy, NR⁴⁶R⁴⁷, S(O)₀₋₂R⁴⁸, C(O)R⁴⁹, CO₂R⁵⁰, C(O)NR⁵¹R⁵², NR⁵³C(O)R⁵⁴, S(O)₂NR⁵⁵R⁵⁶, NR⁵⁷S(O)₂R⁵³ and Z²;
Y represents a bond, OC₁₋₆ alkylene, S(O)₀₋₂C₁₋₆alkylene, N(R⁵⁹)C₁₋₆alkylene or C₁₋₆ alkylene, wherein any alkylene group in Y may be optionally substituted by one or more substituents independently selected from halogen, hydroxy and C₁₋₄ alkoxy;
R²⁹ represents hydrogen, halogen, hydroxyl, C₁₋₆alkoxy, cyano, NR⁶⁰R⁶¹, S(O)₀₋₂R⁶², C(O)R⁶³, CO₂R⁶⁴, C(O)NR⁶⁵R⁶⁶, NR⁶⁷C(O)R⁶⁸, S(O)₂NR⁶⁹R⁷⁰, NR⁷¹S(O)₂R⁷² or Z³; with the proviso that when Y is a bond R²⁹ is not hydrogen;
R⁴², R⁴³, R⁴⁴ and R⁴⁵ each independently represent hydrogen or a C₁₋₆ alkyl group which C₁₋₆ alkyl group may be optionally substituted by one or more substituents independently selected from halogen, hydroxyl, C₁₋₆ alkoxy, NR⁷³R⁷⁴, S(O)₀₋₂R⁷⁵, C(O)R⁷⁶, CO₂R⁷⁷, C(O)NR⁷⁸R⁷⁹, NR⁸⁰C(O)R⁸¹, S(O)₂NR⁸²R⁸³, NR⁸⁴S(O)₂R⁸⁵ or Z⁴, or R⁴⁴ and R⁴⁵, together with the nitrogen atom to which they are both attached, may form a 4-8 membered aliphatic heterocyclic ring, which heterocyclic ring may be optionally substituted by one or more substituents independently selected from halogen, hydroxy, C₁₋₆ alkyl, C₁₋₆ hydroxyalkyl and C₁₋₆ haloalkyl;
Z¹, Z², Z³ and Z⁴ each independently represent tetrazole, or a 5-6 membered heterocyclic ring, which heterocyclic ring is substituted by one or more substituents independently selected from hydroxy, amino, ═O, ═S, and which heterocyclic ring may further be optionally substituted by one or more substituents independently selected from halogen and C₁₋₆ alkyl;
R⁸, R⁹, R¹¹, R¹², R¹³, R¹⁴, R¹⁵, R¹⁶, R¹⁸, R¹⁹, R²⁰, R²¹, R²², R²³, R²⁴, R²⁵, R²⁶, R²⁷, R³⁰, R³², R³³, R³⁴, R³⁵, R³⁶, R³⁷, R³⁸, R³⁹, R⁴⁰, R⁴⁶, R⁴⁷, R⁴⁹, R⁵⁰, R⁵¹, R⁵², R⁵³, R⁵⁴, R⁵⁵, R⁵⁶, R⁵⁷, R⁵⁹, R⁶⁰, R⁶¹, R⁶³, R⁶⁴, R⁶⁵, R⁶⁶, R⁶⁷, R⁶⁸, R⁶⁹, R⁷⁰, R⁷¹, R⁷³, R⁷⁴, R⁷⁶, R⁷⁷, R⁷⁸, R⁷⁹, R⁸⁰, R⁸¹, R⁸², R⁸³ and R⁸⁴ each independently represent hydrogen, C₁₋₆ alkyl, C₁₋₆ hydroxyalkyl or C₁₋₆ haloalkyl; or any of R⁸ and R⁹, R¹¹ and R¹², R¹³ and R¹⁴, R²² and R²³, R²⁵ and R²⁶, R³⁴ and R³⁵, R³⁸ and R³⁹, R⁴⁶ and R⁴⁷, R⁵¹ and R⁵², R⁵⁵ and R⁵⁶, R⁶⁰ and R⁶¹, R⁶⁵ and R⁶⁶, R⁶⁹ and R⁷⁰, R⁷³ and R⁷⁴, R⁷⁸ and R⁷⁹, and R⁸² and R⁸³, together with the nitrogen atom to which they are both attached, may form a 4-8 membered aliphatic heterocyclic ring, which heterocyclic ring may be optionally substituted by one or more substituents independently selected from halogen, hydroxy, C₁₋₆ alkyl, C₁₋₆ hydroxyalkyl and C₁₋₆ haloalkyl;
R¹⁰, R¹⁷, R³¹, R⁴¹, R⁴⁸, R⁵⁸, R⁶², R⁷², R⁷⁵ and R⁸⁵ each independently represent C₁₋₆ alkyl, C₁₋₆ hydroxyalkyl or C₁₋₆ haloalkyl; and
with the proviso that when R¹ is phenyl, D is O, n is 1, R² is hydrogen, R³ is hydrogen, A is NHC(O), p is 0, q is 0 and R⁶ is methyl, then the phenyl group R¹ must be substituted by at least one substituent other than C₁₋₄ alkyl, chlorine and methoxy.

2. A compound according to claim 1 or a pharmaceutically acceptable salt thereof, wherein A represents NHC(O).

3. A compound according to claim 1 or a pharmaceutically acceptable salt thereof, wherein R¹ represents phenyl, which phenyl may be optionally substituted by one or more substituents independently selected from halogen, cyano, nitro, C₁₋₄ alkyl, C₁₋₄ hydroxyalkyl, C₁₋₄ haloalkyl, NR⁸R⁹, S(O)₀₋₂R¹⁰, S(O)₂NR¹¹R¹², C(O)NR¹³R¹⁴, CO₂R¹⁵, NR¹⁶S(O)₂R¹⁷, C(O)R¹⁸, NR¹⁹C(O)R²⁰, NR²¹C(O)NR²²R²³, NR²⁴S(O)₂NR²⁵R²⁶ and OR²⁷,

4. A compound according to claim 1 or a pharmaceutically acceptable salt thereof, wherein D represents NR⁷ and R⁷ represents hydrogen, C₁₋₄ alkyl, C₁₋₄ hydroxyalkyl or C₁₋₄ haloalkyl.

5. A compound according to claim 1 or a pharmaceutically acceptable salt thereof, wherein R⁶ represents a group of formula (II) 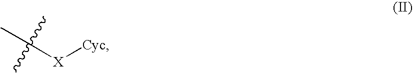
wherein,
X represents a bond; and
Cyc represents a 4-8 membered heterocyclic ring, which heterocyclic ring may be optionally substituted by one or more substituents R²⁸.

6. A compound according to claim 1 or a pharmaceutically acceptable salt thereof, wherein each substituent R²⁸ is independently selected from halogen, OR⁴², NR⁴⁴R⁴⁵ or a C₁₋₄ alkyl group, which C₁₋₄ alkyl group may be optionally substituted by one or more substituents independently selected from halogen, hydroxyl, C₁₋₄ alkoxy, NR⁴⁶R⁴⁷ and C(O)NR⁵¹R⁵²;
R⁴², R⁴⁴ and R⁴⁵ each independently represent hydrogen or a C₁₋₄ alkyl group which C₁₋₄ alkyl group may be optionally substituted by one or more substituents independently selected from halogen, hydroxyl, C₁₋₄ alkoxy, NR⁷³R⁷⁴ and C(O)NR⁷⁸R⁷⁹; and R⁴⁶, R⁴⁷, R⁵¹, R⁵², R⁷³, R⁷⁴, R⁷⁸ and R⁷⁹ each independently represent hydrogene or a C₁₋₄ alkyl group which C₁₋₄ alkyl group may be optionally substituted by one or more substituents independently selected from halogen and hydroxyl.

7. A compound according to claim 1 or a pharmaceutically acceptable salt thereof, wherein R⁶ represents a group of formula (III), 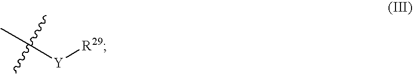
wherein
Y represents a bond, OC₁₋₄alkylene, N(R⁵⁹)C₁₋₄alkylene or C₁₋₄ alkylene, wherein any alkylene group in Y may be optionally substituted by one or more substituents independently selected from halogen, hydroxy and C₁₋₄ alkoxy;
R⁵⁹ represents hydrogen or C₁₋₄ alkyl;
R²⁹ represents hydrogen, halogen, hydroxyl, C₁₋₄alkoxy, NR⁶⁰R⁶¹ and C(O)NR⁶⁵R⁶⁶; and
R⁶⁰, R⁶¹, R⁶⁵ and R⁶⁶ each independently represent hydrogen or a C₁₋₄ alkyl group which C₁₋₄ alkyl group may be optionally substituted by one or more substituents independently selected from halogen and hydroxyl.

8. A compound according to claim 1 of formula (IA), or a pharmaceutically acceptable salt thereof, 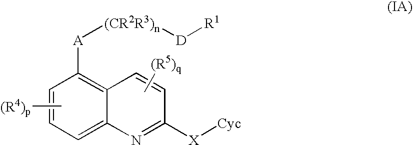
wherein
n is 1;
A represents NHC(O);
D represents O, S or NR⁷, wherein R⁷ represents hydrogen or C₁₋₄ alkyl;
R¹ represents phenyl, which phenyl may be optionally substituted by one or more substituents independently selected from halogen, cyano, nitro, C₁₋₄ alkyl, C₁₋₄ hydroxyalkyl, C₁₋₄ haloalkyl, NR⁸R⁹, S(O)₀₋₂R¹⁰, S(O)₂NR¹¹R¹², C(O)NR¹³R¹⁴, CO₂R¹⁵, NR¹⁶S(O)₂R¹⁷, C(O)R¹⁸, NR¹⁹C(O)R²⁰, NR²¹C(O)NR²²R²³, NR²⁴S(O)₂NR²⁵R²⁶ and OR²⁷;
R² and R³ each independently represent hydrogen or C₁₋₃ alkyl;
p represents 0 or 1; R⁴ represents halogen or C₁₋₄ alkyl;
q represents 0 or 1; R⁵ represents halogen or C₁₋₄ alkyl;
X represents a bond,
Cyc represents a 4-8 membered heterocyclic ring, which heterocyclic ring may be optionally substituted by one or more substituents R²⁸;
each substituent R²⁸ is independently selected from halogen, OR⁴², NR⁴⁴R⁴⁵ or a C₁₋₄ alkyl group, which C₁₋₄ alkyl group may be optionally substituted by one or more substituents independently selected from halogen, hydroxyl, C₁₋₄ alkoxy, NR⁴⁶R⁴⁷ and C(O)NR⁵¹R⁵²;
R⁴², R⁴⁴ and R⁴⁵ each independently represent hydrogen or a C₁₋₄ alkyl group which C₁₋₄ alkyl group may be optionally substituted by one or more substituents independently selected from halogen, hydroxyl, C₁₋₄ alkoxy, NR⁷³R⁷⁴ and C(O)NR⁷⁸R⁷⁹;
R⁴⁶, R⁴⁷, R⁵¹, R⁵², R⁷³, R⁷⁴, R⁷⁸ and R⁷⁹ each independently represent hydrogen or or a C₁₋₄ alkyl group which C₁₋₄ alkyl group may be optionally substituted by one or more substituents independently selected from halogen and hydroxyl;
R⁸, R⁹, R¹¹, R¹², R¹³, R¹⁴, R¹⁵, R¹⁶, R¹⁸, R¹⁹, R²⁰, R²¹, R²², R²³, R²⁴, R²⁵, R²⁶ and R²⁷ each independently represent hydrogen or C₁₋₄ alkyl; and
R¹⁰ and R¹⁷ each independently represent C₁₋₄ alkyl.

9. A compound according to claim 1 of formula (IB), or a pharmaceutically acceptable salt thereof, 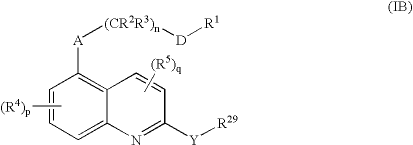
wherein
n is 1;
A represents NHC(O);
D represents O, S or NR⁷, wherein R⁷ represents hydrogen or C₁₋₄ alkyl;
R¹ represents phenyl, which phenyl may be optionally substituted by one or more substituents independently selected from halogen, cyano, nitro, C₁₋₄ alkyl, C₁₋₄ hydroxyalkyl, C₁₋₄ haloalkyl, NR⁸R⁹, S(O)₀₋₂R¹⁰, S(O)₂NR¹¹R¹², C(O)NR¹³R¹⁴, CO₂R¹⁵, NR¹⁶S(O)₂R¹⁷, C(O)R¹⁸, NR¹⁹C(O)R²⁰, NR²¹C(O)NR²²R²³, NR²⁴S(O)₂NR²⁵R²⁶ and OR²⁷;
R² and R³ each independently represent hydrogen or C₁₋₃ alkyl;
p represents 0 or 1; R⁴ represents halogen or C₁₋₄ alkyl;
q represents 0 or 1; R⁵ represents halogen or C₁₋₄ alkyl;
Y represents a bond, OC₁₋₄alkylene, N(R⁵⁹)C₁₋₄alkylene or C₁₋₄ alkylene, wherein any alkylene group in Y may be optionally substituted by one or more substituents independently selected from halogen, hydroxy and C₁₋₄ alkoxy;
R⁵⁹ represents hydrogen or C₁₋₄ alkyl;
R²⁹ represents hydrogen, halogen, hydroxyl, C₁₋₄alkoxy, NR⁶⁰R⁶¹ and C(O)NR⁶⁵R⁶⁶;
with the proviso that when Y is a bond R²⁹ is not hydrogen; and
R⁶⁰, R⁶¹, R⁶⁵ and R⁶⁶ each independently represent hydrogen or a C₁₋₄ alkyl group which C₁₋₄ alkyl group may be optionally substituted by one or more substituents independently selected from halogen and hydroxyl; R⁸, R⁹, R¹¹, R¹², R¹³, R¹⁴, R¹⁵, R¹⁶, R¹⁸, R¹⁹, R²⁰, R²¹, R²², R²³, R²⁴, R²⁵, R²⁶ and R²⁷ each independently represent hydrogen or C₁₋₄ alkyl;
R¹⁰ and R¹⁷ each independently represent C₁₋₄ alkyl and
with the proviso that when R¹ is phenyl, D is O, n is 1, R² is hydrogen, R³ is hydrogen, A is NHC(O), p is 0, q is 0 and YR²⁹ represents methyl, then the phenyl group R¹ must be substituted by at least one substituent other than C₁₋₄ alkyl, chlorine and methoxy.

10. A compound according to claim 1, which is selected from:
N-[6-Chloro-2-[(3R)-3-hydroxy-1-pyrrolidinyl]-5-quinolinyl]-2-(phenylamino)-acetamide,
N-[6-Chloro-2-[(3R)-3-hydroxy-1-pyrrolidinyl]-5-quinolinyl]-2-[(3-chlorophenyl)amino]-acetamide,
N-[6-Chloro-2-[(3R)-3-hydroxy-1-pyrrolidinyl]-5-quinolinyl]-2-[(4-chlorophenyl)amino]-acetamide,
N-[6-Chloro-2-[(3R)-3-hydroxy-1-pyrrolidinyl]-5-quinolinyl]-2-[(2-chlorophenyl)amino]-acetamide,
N-[6-Chloro-2-[(3R)-3-hydroxy-1-pyrrolidinyl]-5-quinolinyl]-2-phenoxy-acetamide,
N-[6-Chloro-2-[(3R)-3-hydroxy-1-pyrrolidinyl]-5-quinolinyl]-2-(phenylthio)-acetamide,
N-[6-Chloro-2-[(3R)-3-hydroxy-1-pyrrolidinyl]-5-quinolinyl]-2-[(3-cyanophenyl)amino]-acetamide,
N-[6-Chloro-2-[(3R)-3-hydroxy-1-pyrrolidinyl]-5-quinolinyl]-2-[(4-cyanophenyl)amino]-acetamide,
N-[6-Chloro-2-[(3R)-3-hydroxy-1-pyrrolidinyl]-5-quinolinyl]-2-[(3-fluorophenyl)amino]-acetamide,
N-[6-Chloro-2-[(3R)-3-hydroxy-1-pyrrolidinyl]-5-quinolinyl]-2-[(4-fluorophenyl)amino]-acetamide,
N-[6-Chloro-2-[(3R)-3-hydroxy-1-pyrrolidinyl]-5-quinolinyl]-2-[(3,4-difluorophenyl)amino]-acetamide,
N-[6-Chloro-2-[(3R)-3-hydroxy-1-pyrrolidinyl]-5-quinolinyl]-2-[(3,4-difluorophenyl)amino]-propanamide,
N-[6-Chloro-2-(1,2-dihydroxypropyl)-5-quinolinyl]-2-[(4-fluorophenyl)amino]-acetamide,
N-(6-Chloro-2-morpholin-4-yl-quinolin-5-yl)-2-(4-fluoro-phenylamino)-acetamide,
N-[6-Chloro-2-(1H-pyrazol-3-yl)-quinolin-5-yl]-2-(4-fluoro-phenylamino)-acetamide or a pharmaceutically acceptable salt thereof.

11. A process for the preparation of a compound of formula (J) according to claim 1 or a pharmaceutically acceptable salt thereof, which comprises either
(a) reacting a compound of formula (IV) 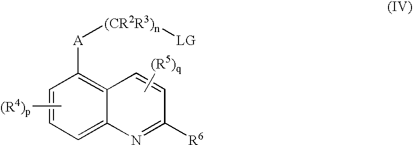
with a compound of formula
HD-R¹ (V)
wherein LG¹ represents a leaving group such as a halogeno or sulphonyloxy group and A, D, R¹, R², R³, R⁴, R⁵, R⁶, n, p and q are as defined in formula (I); or
(b) reacting a compound of formula 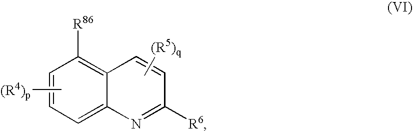
with a compound of formula 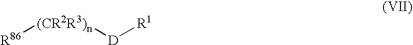
wherein one of R⁸⁶ and R⁸⁷ represents NH₂ and the other of R⁸⁶ and R⁸⁷ represents CO₂H, COF, COBr or COCl, and D, X, R¹, R², R³, R⁴, R⁵, R⁶, n, p and q are as defined in formula (I); or
(c) reacting a compound of formula 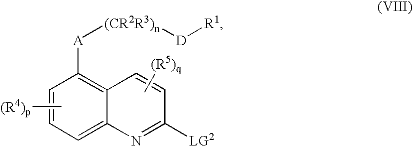
with a compound of formula 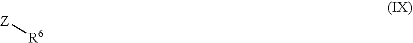
wherein LG² represents a leaving group such as a halogeno or sulphonyloxy group, A, D, R¹, R², R³, R⁴, R⁵, n, p and q are as defined in formula (I), z either represents hydrogen when R⁶ is attached to z at a heteroatom, otherwise when R⁶ is attached to z at a carbon atom, z represents a metallic, organometallic or organosilicon group,
and optionally after (a), (b) or (c), carrying out one or more of the following:
converting the compound to a further compound of the invention
forming a pharmaceutically acceptable salt of the compound.

12. A method of treating a disorder in a subject, the method comprising administering a compound of formula (IC), or a pharmaceutically acceptable salt thereof 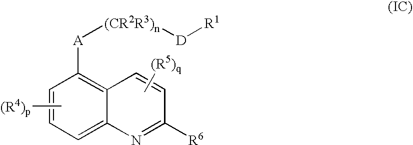
wherein
n is 1, 2 or 3;
when n is 1, A represents NHC(O) or NHC(S);
when n is 2 or 3, A represents NHC(O), C(O)NH, NHC(S) or C(S)NH;
D represents O, S or NR⁷, wherein R⁷ represents hydrogen, C₁₋₆ alkyl, C₁₋₆ hydroxyalkyl or C₁₋₆ haloalkyl;
R¹ represents a 6-10 membered aryl, or a 5-10 membered heteroaryl ring, which aryl or heteroaryl ring may be optionally substituted by one or more substituents independently selected from halogen, cyano, C₁₋₆ alkyl, C₁₋₆ hydroxyalkyl, C₁₋₆ haloalkyl, NR⁸R⁹, S(O)₀₋₂R¹⁰, S(O)₂NR¹¹R¹², C(O)NR¹³R¹⁴, CO₂R¹⁵, NR¹⁶S(O)₂R¹⁷, C(O)R¹⁸, NR¹⁹C(O)R²⁰, NR²¹C(O)NR²²R²³, NR²⁴S(O)₂NR²⁵R²⁶ and OR²⁷;
within each grouping CR²R³, R² and R³ each independently represent hydrogen, halogen, is C₁₋₃ alkyl, C₁₋₃ hydroxyalkyl or C₁₋₃ haloalkyl;
p represents 0, 1, 2 or 3; each R⁴ independently represents halogen, cyano, C₁₋₆ alkyl, C₁₋₆ hydroxyalkyl or C₁₋₆ haloalkyl;
q represents 0, 1 or 2; each R⁵ independently represents halogen, cyano, C₁₋₆ alkyl, C₁₋₆ hydroxyalkyl or C₁₋₆ haloalkyl;
R⁶ represents a group 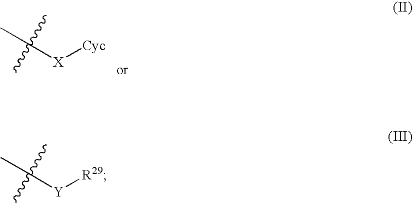
wherein X represents a bond, O, S, NR³⁰ or a C₁₋₄ alkylene which C₁₋₄ alkylene may be optionally substituted by one or more substituents independently selected from halogen, hydroxy and C₁₋₄alkoxy;
Cyc represents a 3-8 membered carbocyclic or a 4-8 membered heterocyclic ring, which ring may be optionally substituted by one or more substituents R²⁸, wherein each substituent R²⁸ is independently selected from halogen, cyano, ═O, S(O)₂R³¹, C(O)R³², CO₂R³³, C(O)NR³⁴R³⁵, NR³⁶C(O)R³⁷, S(O)₂NR³⁸R³⁹, NR⁴⁰S(O)₂R⁴¹, OR⁴², SR⁴³, NR⁴⁴R⁴⁵, Z¹ or a C₁₋₆ alkyl group which C₁₋₆ alkyl group may be optionally be substituted by one or more substituents independently selected from halogen, hydroxyl, C₁₋₆ alkoxy, NR⁴⁶R⁴⁷, S(O)₀₋₂R⁴⁸, C(O)R⁴⁹, CO₂R⁵⁰, C(O)NR⁵¹R⁵², NR⁵³C(O)R⁵⁴, S(O)₂NR⁵⁵R⁵⁶, NR⁵⁷S(O)₂R⁵⁸ and Z²;
Y represents a bond, OC₁₋₆ alkylene, S(O)₀₋₂C₁₋₆alkylene, N(R⁵⁹)C₁₋₆alkylene or C₁₋₆ alkylene, wherein any alkylene group in Y may be optionally substituted by one or more substituents independently selected from halogen, hydroxy and C₁₋₄ alkoxy;
R²⁹ represents hydrogen, halogen, hydroxyl, C₁₋₆alkoxy, cyano, NR⁶⁰R⁶¹, S(O)₀₋₂R⁶², C(O)R⁶³, CO₂R⁶⁴, C(O)NR⁶⁵R⁶⁶, NR⁶⁷C(O)R⁶⁸, S(O)₂NR⁶⁹R⁷⁰, NR⁷¹S(O)₂R⁷² or Z³;
with the proviso that when Y is a bond R²⁹ is not hydrogen;
R⁴², R⁴³, R⁴⁴ and R⁴⁵ each independently represent hydrogen or a C₁₋₆ alkyl group which C₁₋₆ alkyl group may be optionally substituted by one or more substituents independently selected from halogen, hydroxyl, C₁₋₆ alkoxy, NK⁷³R⁷⁴, S(O)₀₋₂R⁷⁵, C(O)K⁷⁶, CO₂R⁷⁷, C(O)NR⁷⁸R⁷⁹, NR⁸⁰C(O)R⁸¹, S(O)₂NR⁸²R⁸³, NR⁸⁴S(O)₂R⁸⁵ or Z⁴, or R⁴⁴ and R⁴⁵, together with the nitrogen atom to which they are both attached, may form a 4-8 membered aliphatic heterocyclic ring, which heterocyclic ring may be optionally substituted by one or more substituents independently selected from halogen, hydroxy, C₁₋₆ alkyl, C₁₋₆ hydroxyalkyl and C₁₋₆ haloalkyl;
Z¹, Z², Z³ and Z⁴ each independently represent tetrazole, or a 5-6 membered heterocyclic ring, which heterocyclic ring is substituted by one or more substituents independently selected from hydroxy, amino, ═O, ═S, and which heterocyclic ring may further be optionally substituted by one or more substituents independently selected from halogen and C₁₋₆ alkyl;
R⁸, R⁹, R¹¹, R¹², R¹³, R¹⁴, R¹⁵, R¹⁶, R¹⁸, R¹⁹, R²⁰, R²¹, R²², R²³, R²⁴, R²⁵, R²⁶, R²⁷, R³⁰, R³², R³³, R³⁴, R³⁵, R³⁶, R³⁷, R³⁸, R³⁹, R⁴⁰, R⁴⁶, R⁴⁷, R⁴⁹, R⁵⁰, R⁵¹, R⁵², R⁵³, R⁵⁴, R⁵⁵, R⁵⁶, R⁵⁷, R⁵⁹, R⁶⁰, R⁶¹, R⁶³, R⁶⁴, R⁶⁵, R⁶⁶, R⁶⁷, R⁶⁸, R⁶⁹, R⁷⁰, R⁷¹, R⁷³, R⁷⁴, R⁷⁶, R⁷⁷, R⁷⁸, R⁷⁹, R⁸⁰, R⁸¹, R⁸², R⁸³ and R⁸⁴ each independently represent hydrogen, C₁₋₆ alkyl, C₁₋₆ hydroxyalkyl or C₁₋₆ haloalkyl; or any of R⁸ and R⁹, R¹¹ and R¹², R¹³ and R¹⁴, R²² and R²³, R²⁵ and R²⁶, R³⁴ and R³⁵, R³⁸ and R³⁹, R⁴⁶ and R⁴⁷, R⁵¹ and R⁵², R⁵⁵ and R⁵⁶, R⁶⁰ and R⁶¹, R⁶⁵ and R⁶⁶, R69 and R⁷⁰, R⁷³ and R⁷⁴, R⁷⁸ and R⁷⁹, and R⁸² and R⁸³, together with the nitrogen atom to which they are both attached, may form a 4-8 membered aliphatic heterocyclic ring, which heterocyclic ring may be optionally substituted by one or more substituents independently selected from halogen, hydroxy, C₁₋₆ alkyl, C₁₋₆ hydroxyalkyl and C₁₋₆ haloalkyl; and R¹⁰, R¹⁷, R³¹, R⁴¹, R⁴⁸, R⁵⁸, R⁶², R⁷², R⁷⁵ and R⁸⁵ each independently represent C₁₋₆ alkyl, C₁₋₆ hydroxyalkyl or C₁₋₆ haloalkyl.

13. A pharmaceutical composition comprising a compound of formula (IC), or a pharmaceutically acceptable salt thereof, as defined in claim 12 in association with a pharmaceutically acceptable adjuvant, diluent or carrier.

14. A process for the preparation of a pharmaceutical composition comprising a compound of formula (IC), or a pharmaceutically acceptable salt thereof, as defined in claim 12 in association with a pharmaceutically acceptable adjuvant, diluent or carrier, wherein the process comprises mixing a compound of formula (IC), or a pharmaceutically acceptable salt thereof, as defined in claim 12 with a pharmaceutically acceptable adjuvant, diluent or carrier.

15. A method of treating rheumatoid arthritis, the method comprising administering a compound of formula (IC), or a pharmaceutically acceptable salt thereof, as defined in claim 12.

16. A method of treating osteoarthritis, the method comprising administering a compound of formula (IC), or a pharmaceutically acceptable salt thereof, as defined in claim 12.

17. A method of treating asthma or chronic obstructive pulmonary disease, the method comprising administering a compound of formula (IC), or a pharmaceutically acceptable salt thereof, as defined in claim 12.

18. A method of treating inflammatory bowel disease, the method comprising administering a compound of formula (IC), or a pharmaceutically acceptable salt thereof, as defined in claim 12.
